(19) United States
(12) Patent Application Publication
(10) Pub. No.: US 2021/0251654 A1
(43) Pub. Date: Aug. 19, 2021

(54) SURGICAL SMOKE AND GASES VENTING CANNULA ATTACHMENT

(71) Applicant: Fisher & Paykel Healthcare Limited, Auckland (NZ)

(72) Inventors: Benjamin Elliot Hardinge Pegman, Auckland (NZ); Christian Francis Fischer, Auckland (NZ); Zane Paul Gell, Auckland (NZ); Vincent Verdoold, Auckland (NZ); Richard John Boyes, Auckland (NZ); Katie-Ann Jane Buckels, Auckland (NZ); Charlotte Grace Laus, Auckland (NZ); Abigail Sharmini Rajen Arulandu, Auckland (NZ); Zach Jonathan Warner, Auckland (NZ)

(21) Appl. No.: 17/269,404

(22) PCT Filed: Aug. 21, 2019
(86) PCT No.: PCT/NZ2019/050103

§ 371 Date: Feb. 18, 2021

Related U.S. Application Data
(60) Provisional application No. 62/720,687, filed on Aug. 21, 2018.

(51) Int. Cl. A61B 17/34; A61M 13/00

(52) U.S. Cl. CPC A61B 17/3421; A61B 2017/00477; A61B 2217/005; A61M 13/003

(57) ABSTRACT

Various venting attachments or leak devices can be removably or permanently coupled to a surgical cannula to allow a user to vent gases, in particular smoke, from a surgical cavity. The venting attachment can be configured to vent the smoke at a predetermined rate and filter out the smoke prior to venting to atmosphere. The substantially constant venting flow rate can promote clearing of the smoke in the surgical cavity while helping to maintain a substantially constant pressure, and thus stability, in the surgical cavity.

### FIELD OF THE DISCLOSURE

The present disclosure relates to humidifier systems and components of humidifier systems configured to supply gases to a patient, in particular to venting of surgical gases and/or smoke from a body cavity in a patient.

### BACKGROUND

Various medical procedures require the provision of gases (for example, heated gases), typically carbon dioxide, to a patient during the medical procedure, for example, closed type medical procedures and open type medical procedures.

In closed type medical procedures, an insufflator is arranged to deliver gases to a body cavity of the patient to inflate the body cavity and/or to resist collapse of the body cavity during the medical procedure. Examples of such medical procedures include laparoscopy and endoscopy, although an insufflator may be used with any other type of medical procedure as required. Endoscopic procedures enable a medical practitioner to visualize a body cavity by inserting an endoscope or the like through natural openings or small puncture(s) to generate an image of the body cavity. In laparoscopy procedures, a medical practitioner typically inserts a surgical instrument through one or more natural openings, small puncture(s), or incision(s) to perform a medical procedure in the body cavity. In some cases an initial endoscopic procedure may be carried out to assess the body cavity, and then a subsequent laparoscopy carried out to operate on the body cavity. Such procedures are widely used, for example, within the peritoneal cavity, or during a thoracoscopy, colonoscopy, gastroscopy or bronchoscopy.

In open type medical procedures, for example, open surgeries, gases are used to fill a surgical cavity, with excess gases spilling outward from the opening. The gases can also be used to provide a layer of gases over exposed body parts, for example, including internal body parts, where there is no discernible cavity. For these procedures, rather than serving to inflate a cavity, the gases can be used to prevent or reduce desiccation and infection by covering exposed internal body parts with a layer of heated, humidified, sterile gases.

An apparatus for delivering gases during these medical procedures can include an insufflator arranged to be connected to a remote source of pressurized gases, for example, an insufflation fluid (for example, gases) supply system in a hospital. The apparatus can be operative to control the pressure and/or flow of the gases from the gases source to a level suitable for delivery into the body cavity, usually via a cannula or needle connected to the apparatus and inserted into the body cavity, or via a diffuser arranged to diffuse gases over and into the wound or surgical cavity. In many cases, a humidifier is operatively coupled to the insufflator. A controller of the apparatus can energize a heater of the humidifier located in the gases flow path to deliver humidification fluid (for example, water vapor) to the insufflation fluid (for example, gases) stream prior to entering the patient's body cavity. The humidified insufflation fluid can be delivered to the patient via further tubing which may also be heated. The insufflator and humidifier can be located in separate housings that are connected together via suitable tubing and/or electrical connections, or located in a common housing arranged to be connected to a remote gas supply via suitable tubing.

The internal body temperature of a human patient is typically around 37° C. It can be desirable to match the temperature of the gases delivered from the apparatus as closely as possible to the typical human body temperature. It can also be desirable to deliver gases above or below internal body temperature, such as, for example, 1, 2, 3, 4, 5, 6, 7, 8, 9, 10, or 15 degrees above or below internal body temperature for example, or ranges including any two of the foregoing values. It can also be desirable to deliver gases of a desired fixed or variable humidity and/or a desired fixed or variable gas temperature (which may also be referred to herein as standard), such as dry cold gas, dry hot gas, humidified cold gas, or humidified hot gas for example. Further, the gases delivered into the patient's body can be relatively dry, which can cause damage to the body cavity, including cell death or adhesions or cell desiccation. In many cases, a humidifier is operatively coupled to the insufflator. A controller of the apparatus can energize a heater of the humidifier located in the gases flow path to deliver humidification fluid to the insufflation fluid (for example, gases) stream prior to entering the patient's body cavity.

The humidified gas can be delivered to the patient via further tubing which may also be heated. The insufflator and humidifier can be located in separate housings that are connected together via suitable tubing and/or electrical connections, or located in a common housing arranged to be connected to a remote gas supply via suitable tubing.

### SUMMARY

During certain surgical procedures (for example, laparoscopic surgery, or other procedures disclosed herein), electrosurgery, electrocautery, energy and laser cutting and/or cauterizing, among others, is used to cause cutting and/or coagulation of tissue (including other organs, anatomical structures, and/or the like) and/or blood vessels within the surgical cavity (for example, a pneumoperitoneum). Smoke can be produced by electrosurgery, electrocautery, energy and laser cutting and/or cauterizing, among others. The concentration of smoke can increase over time, particularly in a sealed and pressurized surgical cavity and/or when there are no significant gases leaks, suction, and/or irrigation. The smoke plume rises and can block vision. Further the smoke plume may contact or deposit particles on the scope. The smoke plume contacting the scope can also cause fogging or condensation on the scope. Condensation can occur on various surfaces on a medical instrument. When condensation forms on a viewing surface of a medical instrument, this is observed as a fogging effect which manifests as an impairment of visibility through a lens or any other viewing surface of a medical instrument (such as, for example, a mirror or transparent or translucent window). When condensation forms on various surfaces of a medical instrument, the condensation can coalesce into water droplets. This can occur directly on the viewing surface or other surfaces which can then migrate to or be deposited on the viewing surface. Accordingly, as used herein condensation and/or fogging means condensation generally and in some instances, specifically with respect to condensation on a viewing surface (i.e. fogging).

The smoke can impede vision, for example, vision of a surgeon or other medical personnel participating in the medical procedure (for example, surgery) when the medical personnel views the surgical space via a camera inserted through the cannula. Without the use of venting or suction, the medical personnel need to release the gases and/or smoke from inside the surgical cavity through deflation and re-insufflation of the surgical cavity.

In some embodiments, systems and methods can advantageously filter to remove harmful chemicals & bio-particles from the gas being vented into the operation room. A growing concern in surgical environments is hazards to surgical staff (and patients) from the smoke produced during electrosurgery, electrocautery, laser and other energy-based cutting and/or cauterizing, among others. Surgical smoke may contribute to o cancers or other health issues, and contains many chemicals and bio-particles that can be hazardous for human inhalation. In some embodiments, systems and methods as disclosed herein can advantageously vent gas (and smoke plumes created during electrosurgery, electrocautery, laser and other energy-based cutting and/or cauterizing, among others) from inside the pneumoperitoneum which achieves at least two advantages: it can dilute the smoke concentration inside the pneumoperitoneum to improve visibility, and it can also ensure a constant flow of CO₂ from the insufflator which creates an airflow of “clean” CO₂ gas across/over the viewing area which carries or pushes away smoke that is hindering vision in the area between the camera and operating area. The venting flow rate may be related to the delivered flow rate. In one example, the venting rate (i.e. venting flow rate) may be set to achieve a specified pressure within the surgical site. Explained another way, the venting rate is such that a surgical cavity is maintained at a predetermined pressure rate. In some embodiments, venting flow rate may be a predetermined flow rate. For example, the flow rate may be set by the user. The venting element used may be constructed or tuned to achieve the predetermined flow rate. However, there can be a trade-off when venting in terms of negatively affecting stability and pressure of the pneumoperitoneum.

The present disclosure provides examples of venting attachments and/or leak devices (for example, for attaching to or used in combination with a cannula) for venting surgical smoke and/or gases from a surgical cavity. The venting attachment examples disclosed herein can be used with a directed flow cannula, or within a system that includes a supplementary gases source and the directed flow cannula to help with visualization.

The directed flow cannula can include a guiding element that guides the medical device (for example, a scope) in the cannula such that gases surround the scope at and beyond the outlet of the cannula. The guiding element can prevent flow non-uniformity and can prevent the scope from resting against the wall of the cannula outlet. This prevents flow non-uniformity (which may also be referred to as stagnation zones).

Alternatively, the venting attachment is coupled to a separate cannula that is used as a venting cannula. The venting cannula is standard cannula with the venting attachment attached thereto. The venting attachment is spaced apart and separate from a cannula that delivers gases into the surgical cavity.

In some configurations, the venting attachment can include a flow control structure. The flow control structure is shaped and dimensioned in order to control the venting flow rate in order to maintain pressure within the surgical cavity.

In some configurations, the leak device can control a venting flow rate. The venting rate is controlled by an active venting device or be a passive venting rate. In some configurations, the venting rate can match the delivered flow rate. Alternatively, the venting rate can exceed or be less than an insufflation fluid flow rate. The leak device is configured to vent at a rate such that over-pressurization of the surgical cavity is reduced and/or prevented. Preferably the leak device can prevent over-pressurization of surgical cavity, for example, the pneumoperitoneum.

The venting can dilute the smoke concentration inside the surgical cavity to improve visibility and promote a substantially constant flow of carbon dioxide (or other insufflation fluid) from the insufflator, which can produce a flow of cleaner gases across and over the viewing area to push and/or carry away surgical smoke in the area between the camera lens and the operating area.

In some configurations, the venting attachments and/or leak devices disclosed herein can reduce smoke concentration within the surgical cavity while maintaining a substantially stable surgical cavity, for example, by maintaining a substantially stable pressure within the surgical cavity.

In some configurations, the venting attachment and/or leak devices can include a smoke filter so that the vented gases and/or smoke can contain less potentially hazardous particles and/or chemicals.

In some configurations, the leak device examples disclosed herein can include a heating element to heat the vented gases and/or the filter so as to reduce and/or prevent condensation or clogging in the filter, which can improve the lifetime and efficiency of the filter.

In some configurations, the vented gases paths in the leak device examples can include valves to control a leak rate.

In some configurations, the venting attachment is used with a cannula that includes one or more heater elements. The venting attachment may also include a heating element. The heat can prevent condensation of the vented gases.

In some configurations, the venting attachment can include a connector that includes a flow control device. The flow control device is configured to control the venting flow rate. The flow control device may be structured to allow a venting rate that maintains a predetermined pressure within the surgical cavity.

In some configurations, an example leak device or venting attachment to a surgical cannula for venting gases and/or smoke from a surgical cavity can include a cannula connecting component configured to connect to a portion of the surgical cannula inserted into the surgical cavity. The venting attachment can also include a venting component configured to vent gases and/or smoke from the surgical cavity at a predetermined rate. The venting attachment can also include a venting gases pathway extending from the cannula or the surgical cavity to the venting component. The venting attachment can also include a filter component configured to filter the gases and/or smoke before leaving the venting component, wherein the filter component is located in the venting gases pathway and adjacent the venting component.

In some configurations, an example venting attachment to a surgical cannula for venting gases and/or smoke from a surgical cavity is provided, wherein the surgical cannula can comprise a gases port. The venting attachment can include a cannula connecting component configured to connect to a portion of the surgical cannula inserted into the surgical cavity. The venting attachment can also include a venting component configured to vent gases and/or smoke from the surgical cavity at a predetermined rate, an orifice of the cannula connecting component having a size that allows the venting at the predetermined rate. The venting attachment can also include a venting gases pathway extending from the cannula or the surgical cavity to the venting component. The venting attachment can also include a filter component configured to filter the gases and/or smoke before leaving the venting component, wherein the filter component is located in the venting gases pathway and adjacent the venting component.

In some configurations, the filter component can comprise an ULPA filter.

In some configurations, the filter component can further comprise a carbon filter.

In some configurations, the filter component can comprise a filter housing configured to reduce or absorb humidity or condensation in the filters.

In some configurations, the filter housing can comprise water traps and/or a desiccant.

In some configurations, the venting attachment can comprise a heating element.

In some configurations, the heating element is configured to heat the gases and/or the filter to reduce condensation and/or clogging of the filter component.

In some configurations, the heating element can be configured to heat the gases and/or the filter to reduce and/or prevent condensation and/or clogging of the filter component.

In some configurations, the heating element can comprise a flexible PCB heater, a PCB heater, a heater wire, multiple heater wires, or conductive ink. In some configurations, the heating element can comprise a flexible PCB heater, a PCB heater, a heater wire, multiple heater wires, conductive ink, or conductive polymer.

In some configurations, the heating element is in electrical communication with and powered by a controller of an insufflator or a humidifier unit. In some configurations, the heating element is in electrical communication with and powered by an external controller.

In some configurations, the venting component can comprise small orifices.

In some configurations, the venting component can comprise one or more valves in the venting gases pathway.

In some configurations, the one or more valves are mechanically actuated. In some configurations, the one or more valves are electrically actuated.

In some configurations, the one or more valves can comprise a solenoid valve.

In some configurations, the one or more valves are in electrical communication with a controller of an insufflator or a humidifier unit. In some configurations, the one or more valves are in electrical communication with an external controller.

In some configurations, the one or more valves are selectively opened or closed to control a vent rate.

In some configurations, the venting component is configured to vent the gases and/or smoke at a rate that is greater than or equal to a gases delivery flow rate. In some configurations, the venting component is configured to vent the gases and/or smoke at a rate that is equal to the gases delivery rate. In some configurations, the venting component is configured to vent the gases and/or smoke at a rate that is less than the gases delivery rate.

In some configurations, the venting component is configured to maintain pressure inside the surgical cavity at or below 50 mmHg.

In some configurations, the leak device or venting attachment can comprise a flow and/or smoke sensor located in the venting gases pathway.

In some configurations, the cannula connecting component is configured to be connected to a gases port of the cannula.

In some configurations, the venting component can comprise a leak device including a flow restriction. The flow restriction is shaped and dimensioned to define a venting flow rate.

In some configurations, the venting component can comprise a shape that increases in cross-sectional dimension distally with respect to proximally. In some configurations, the venting component can comprise the cross-sectional diameter greater at a distal end than at a proximal end. In some configurations, the venting component can comprise an elongate and flexible shape. In some configurations, the venting component can comprise a housing. In some configurations, the housing can be rectangular, round, curved, elliptical, or polygonal.

In some configurations, the venting component can comprise a user display configured to display a vent rate and/or gases composition.

In some configurations, the venting component can comprise a suction port. In some configurations, the venting component can comprise a manual pump.

In some configurations, the venting attachment is configured to receive gases from the cannula.

In some configurations, the cannula connecting component is configured to be connected to at least a portion of an elongate shaft of the cannula.

In some configurations, the cannula connecting component can comprise a sleeve or seal configured to be coupled near a distal end of the cannula. In some configurations, the cannula connecting component can comprise a sleeve extending along substantially an entire length of the elongate shaft.

In some configurations, the leak device or venting attachment can comprise at least one distal opening and at least one proximal opening. When in use, the at least one distal opening is located inside the surgical cavity and the at least one proximal opening is located outside the surgical cavity.

In some configurations, the cannula connecting component is configured to be connected to an upper housing of the cannula.

In some configurations, the cannula connecting component can comprise a sleeve cap configured to be coupled to the upper housing and enclose the gases port with a gases impermeable lining.

In some configurations, the cannula connecting component can comprise a sleeve portion and the venting component comprises a base coupled to the sleeve portion.

In some configurations, the base is above the upper housing of the cannula when the attachment is coupled to the cannula.

In some configurations, the sleeve portion can comprise at least one opening configured to allow insufflation gases received from the gases port to enter a lumen of the cannula.

In some configurations, the cannula connecting component can comprise a hinged device including two halves connected by a hinge mechanism. The hinged device is configured to clamp around a portion of the upper housing of the cannula.

In some configurations, the venting attachment or leak device can further comprise a rubber seal configured to seal around the gases port of the cannula. The rubber seal can comprise a gases path configured to guide the venting gases and/or smoke to the filter.

In some configurations, the cannula is configured to deliver insufflation gases to the surgical cavity and/or to remove the gases from the surgical cavity.

In some configurations, the cannula connecting component can comprise a Luer lock connector that forms a seal around an outer surface of a gases port of the surgical cannula when the cannula connecting component and the gases port are coupled.

In some configurations, the seal between the cannula connecting component and the gases port can be the only seal between the cannula connecting component and the gases port.

In some configurations, the cannula connecting component can be configured to receive and guide the gases port during insertion into the cannula connecting component.

In some configurations, the cannula connecting component can comprise an opening, a neck region, and/or a confined area.

In some configurations, the neck region can be immediately adjacent the opening and the confined area.

In some configurations, the neck region can be configured to deform to allow passage of the gases port.

In some configurations, the gases port can comprise a flanged end portion and the neck region is configured to deform to allow passage of the flanged end portion.

In some configurations, the confined area can be configured to receive and retain the gases port when the cannula connecting component and the gases port are coupled.

In some configurations, the gases port can comprise a flanged end portion and the confined area is configured to receive and retain the flanged end portion when the cannula connecting component and the gases port are coupled.

In some configurations, the neck region can conform around an outer surface of the gases port to form the seal when the cannula connecting component and the gases port are coupled.

In some configurations, the seal can be formed only between the neck region and the outer surface of the gases port.

In some configurations, the gases port can comprise a shaft portion and the neck region conforms around an outer surface of the shaft portion to form the seal when the cannula connecting component and the gases port are coupled.

In some configurations, the seal can be provided along a length of the shaft portion.

In some configurations, the venting component can be configured to vent the gases and/or smoke at a rate between 1 L/min and 10 L/min.

In some configurations, the venting component can be configured to vent the gases and/or smoke at a rate between 5 L/min and 7 L/min.

In some configurations, the venting component can be configured to vent the gases and/or smoke at a rate of 4 L/min.

In some configurations, a venting attachment to a surgical cannula for venting gases and/or smoke from a surgical cavity can comprise a venting component configured to vent gases and/or smoke from the surgical cavity at a predetermined rate; a venting gases pathway extending from the cannula or the surgical cavity to the venting component; and a filter component configured to filter the gases and/or smoke before leaving the venting component, wherein the filter component can be located in the venting gases pathway and adjacent the venting component, wherein the venting component can be at least partially inserted through a surgical opening leading to the surgical cavity.

In some configurations, the venting attachment can comprise a cannula connecting component configured to connect to a portion of the surgical cannula inserted into the surgical cavity through the surgical opening.

In some configurations, the cannula connecting component can be configured to be connected to a gases port of the cannula.

In some configurations, the filter component can comprise an ULPA filter.

In some configurations, the filter component further can comprise a carbon filter.

In some configurations, the filter component can comprise a filter housing configured to reduce or absorb humidity or condensation in the filter.

In some configurations, the filter housing can comprise water traps and/or a desiccant.

In some configurations, the venting attachment can comprise a heating element.

In some configurations, the heating element can be configured to heat the gases and/or the filter to reduce and/or prevent condensation and/or clogging of the filter component.

In some configurations, the heating element can comprise a flexible PCB heater, a PCB heater, a heater wire, multiple heater wires, conductive ink, or conductive polymers.

In some configurations, the heating element can be in electrical communication with and powered by a controller of an insufflator or a humidifier unit or an external controller.

In some configurations, the venting component can comprise small orifices.

In some configurations, the venting component can comprise one or more valves in the venting gases pathway.

In some configurations, the one or more valves can be mechanically actuated.

In some configurations, the one or more valves can be electrically actuated.

In some configurations, the one or more valves can comprise a solenoid valve.

In some configurations, the one or more valves can be in electrical communication with a controller of an insufflator or a humidifier unit or an external controller.

In some configurations, the one or more valves can be selectively opened or closed to control a vent rate.

In some configurations, the venting component can be configured to vent the gases and/or smoke at a rate that is greater than or equal to a gases delivery flow rate.

In some configurations, the venting component can be configured to vent the gases and/or smoke at a rate that is less than or equal to the gases delivery rate.

In some configurations, the venting component can be configured to maintain pressure inside the surgical cavity at or below 50 mmHg.

In some configurations, the venting component can comprise a leak device including a flow restriction, the flow restriction shaped and dimensioned to define a venting flow rate.

In some configurations, the venting component can comprise an elongate shape.

In some configurations, the venting attachment can comprise a flow and/or smoke sensor located in the venting gases pathway.

In some configurations, a venting attachment to a surgical cannula for venting gases and/or smoke from a surgical cavity can be provided, wherein the surgical cannula can comprise a gases port and an inlet. The venting attachment can comprise a venting component configured to be inserted through the inlet of the surgical cannula, the venting component configured to vent gases and/or smoke from the surgical cavity at a predetermined rate; a venting gases pathway extending from the cannula or the surgical cavity to the venting component; and a filter component configured to filter the gases and/or smoke before leaving the venting component, wherein the filter component can be located in the venting gases pathway.

In some configurations, the venting gases pathway can extend from a distal end of the cannula or the surgical cavity to the inlet of the cannula.

In some configurations, the venting component can be configured to allow passage of insufflation gases into the surgical cavity.

In some configurations, the venting component can be configured to allow passage of one or more medical instruments.

In some configurations, the venting component can be coupled to an upper housing of the surgical cannula.

In some configurations, the venting component can be removably coupled to an upper housing of the surgical cannula.

In some configurations, the venting component can comprise a sleeve portion and a base coupled to the sleeve portion.

In some configurations, the base can be above the upper housing of the surgical cannula when the venting component is coupled to the surgical cannula.

In some configurations, the sleeve portion can comprise at least one opening configured to allow insufflation gases received from the gases port to enter a lumen of the cannula.

In some configurations, the filter component can comprise an ULPA filter.

In some configurations, the filter component further can comprise a carbon filter.

In some configurations, the filter component can comprise a filter housing configured to reduce or absorb humidity or condensation in the filters.

In some configurations, the filter housing can comprise water traps and/or a desiccant.

In some configurations, the venting component can comprise one or more valves in the venting gases pathway.

In some configurations, the one or more valves can be mechanically actuated.

In some configurations, the one or more valves can be electrically actuated.

In some configurations, the one or more valves can comprise a solenoid valve.

In some configurations, the one or more valves can be in electrical communication with a controller of an insufflator or a humidifier unit or an external controller.

In some configurations, the one or more valves can be selectively opened or closed to control a vent rate.

In some configurations, the venting component can be configured to vent the gases and/or smoke at a rate that is greater than or equal to a gases delivery flow rate.

In some configurations, the venting component can be configured to vent the gases and/or smoke at a rate that is less than or equal to the gases delivery rate.

In some configurations, the venting component can be configured to maintain pressure inside the surgical cavity at or below 50 mmHg.

In some configurations, a venting attachment to a surgical cannula for venting gases and/or smoke from a surgical cavity can be provided, wherein the surgical cannula can comprise a gases port, the surgical cannula further having a proximal end and a distal end, the proximal end including an inlet and the distal end of the surgical cannula configured to be inserted into the surgical cavity. The venting attachment can comprise a cannula connecting component configured to connect to the gases port or an inlet of the surgical cannula; a venting component configured to vent gases and/or smoke from the surgical cavity at a predetermined rate; a venting gases pathway extending from the cannula or the surgical cavity to the venting component; and a filter component configured to filter the gases and/or smoke before leaving the venting component, wherein the filter component can be located in the venting gases pathway and adjacent the venting component, wherein the venting component can at least partially surround the proximal end of the surgical cannula.

In some configurations, the filter component can comprise an ULPA filter.

In some configurations, the filter component further can comprise a carbon filter.

In some configurations, the filter component can comprise a filter housing configured to reduce or absorb humidity or condensation in the filters.

In some configurations, the filter housing can comprise water traps and/or a desiccant.

In some configurations, the cannula connecting component can comprise a hinged device including two components connected by a hinge mechanism.

In some configurations, the hinged device can be configured to clamp around a portion of the proximal end of the surgical cannula.

In some configurations, the venting attachment can further comprise a rubber seal configured to seal around the gases port of the cannula, the rubber seal comprising a gases path configured to guide the venting gases and/or smoke to the filter.

In some configurations, the venting attachment can comprise a heating element.

In some configurations, the heating element can be configured to heat the gases and/or the filter to reduce and/or prevent condensation and/or clogging of the filter component.

In some configurations, the heating element can comprise a flexible PCB heater, a PCB heater, a heater wire, multiple heater wires, conductive ink, or conductive polymers.

In some configurations, the heating element can be in electrical communication with and powered by a controller of an insufflator or a humidifier unit or an external controller.

In some configurations, the venting component can comprise small orifices.

In some configurations, the venting component can comprise one or more valves in the venting gases pathway.

In some configurations, the one or more valves can be mechanically actuated.

In some configurations, the one or more valves can be electrically actuated.

In some configurations, the one or more valves can comprise a solenoid valve.

In some configurations, the one or more valves can be in electrical communication with a controller of an insufflator or a humidifier unit or an external controller.

In some configurations, the one or more valves can be selectively opened or closed to control a vent rate.

In some configurations, the venting component can be configured to vent the gases and/or smoke at a rate that is greater than or equal to a gases delivery flow rate.

In some configurations, the venting component can be configured to vent the gases and/or smoke at a rate that is less than or equal to the gases delivery rate.

In some configurations, the venting component can be configured to maintain pressure inside the surgical cavity at or below 50 mmHg.

In some configurations, the venting component can be a permeable or selectively permeable membrane to the filtered gases.

In some configurations, an example venting attachment to a surgical cannula for venting gases and/or smoke from a surgical cavity can comprise a leak device including a flow restriction within a passage of the leak device. The flow restriction is shaped and dimensioned to control a venting flow rate such that the venting flow rate is equal to or less than the flow rate of gases delivered into the surgical cavity. The leak device can further comprise a cannula connector.

In some configurations, an example surgical cannula for providing insufflation gases to a surgical cavity and venting from the surgical cavity can comprise an upper housing including an opening. The cannula can comprise an elongate shaft extending from the upper housing. The cannula can comprise a first lumen in the elongate shaft configured to receive the insufflation gases from a gases source. The cannula can comprise a second lumen in the elongate shaft configured to vent gases from the surgical cavity. The first and second lumens is in fluidic communication with the opening. The cannula can comprise a leak device. The leak device can comprise a cannula connecting component configured to connect to a portion of the cannula. The leak device can comprise a venting component configured to allow gases and/or smoke to exit the surgical cavity. The leak device can comprise a venting gases pathway extending from the cannula to the venting component. The venting gases pathway is in fluidic communication with the second lumen. The leak device can comprise a filter component configured to filter the gases and/or smoke before leaving the venting end. The filter component is located in the venting gases pathway and adjacent the venting component.

In some configurations, the filter component can comprise an ULPA filter.

In some configurations, the filter component can further comprise a carbon filter.

In some configurations, the filter component can comprise a filter housing configured to reduce or absorb humidity or condensation in the filters.

In some configurations, the filter housing can comprise water traps and/or a desiccant.

In some configurations, the cannula can comprise at least one heating element.

In some configurations, the at least one heating element is configured to heat the insufflation gases received from the gases source.

In some configurations, the at least one heating element is located in the leak device. In some configurations, the at least one heating element is located along or within a portion of the upper housing or elongate shaft.

In some configurations, the at least one heating element is configured to heat the vented gases and/or the filter.

In some configurations, the at least one heating element can comprise a flexible PCB heater, a PCB heater, a heater wire, multiple heater wires, or conductive ink. In some configurations, the at least one heating element can comprise a flexible PCB heater, a PCB heater, a heater wire, multiple heater wires, conductive ink, or conductive polymers.

In some configurations, the first and second lumens is offset from each other. In some embodiments, the first and second lumens is concentric.

In some configurations, the first and second lumens is of the same diameter. In some configurations, the first and second lumens is of different diameters.

In some configurations, the second lumen can have a smaller diameter than the first lumen.

In some configurations, an example method of venting gases from a body cavity can comprise inserting a cannula into the body cavity, the cannula comprising a venting attachment, wherein the body cavity is configured to receive an insufflation gas from said cannula or another cannula; filtering the insufflation gases and/or surgical smoke through a filter within the venting attachment; and venting the filtered insufflation gases and/or surgical smoke from the body cavity through the venting attachment.

In some configurations, the method can further comprise heating the gases and/or surgical smoke and/or the filter using a heating element disposed on or within the venting attachment.

In some configurations, the venting is at a rate that is greater than or equal to a gases delivery flow rate.

In some configurations, the venting is at a rate that is equal to the gases delivery rate.

In some configurations, the venting is at a rate that is less than the gases delivery rate.

In some configurations, the venting is configured to maintain pressure inside the surgical cavity at or below 50 mmHg.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure. In some cases, a “slice” has been shown for clarity purposes for some sectional and cross-sectional views of a three dimensional cannula. A person reasonably skilled in the art would be able to appreciate from the disclosure herein that these figures illustrate a slice of a three dimensional cannula. Certain features may not be shown in the slices, for example, any projected surfaces including but not limited to hole surface projections. A person reasonably skilled in the art would be able to appreciate from the disclosure herein that the three dimensional cannula with such slices can include those features.

FIG. 1 illustrates schematically an example medical gases delivery system in use in surgery.

FIGS. 2A-2D illustrate schematically examples of a medical gases delivery system.

FIG. 3 illustrates schematically of an example medical gases delivery cannula system with an example suspended filter device attached to a venting cannula on a patient.

FIG. 3A schematically a cross-sectional view of a venting flow control device.

FIG. 3B illustrates schematically a front view of the venting cannula of FIG. 3.

FIG. 3C illustrates schematically a cross-sectional view of the suspended filter device.

FIG. 3D illustrates schematically filter connections of the suspended filter leak device.

FIG. 3E illustrates schematically a cross-sectional view of the suspended leak device connected to a double-lumen cannula.

FIG. 4 illustrates schematically an example medical gases delivery cannula system with another example suspended filter device attached to a venting cannula on a patient.

FIG. 4A illustrates schematically a front view of the venting cannula of FIG. 4.

FIG. 4B illustrates schematically a cross-sectional view of the suspended filter device.

FIG. 5 illustrates schematically of an example medical gases delivery cannula system with a rectangular-shaped filter device attached to a venting cannula on a patient.

FIG. 5A illustrates schematically a front view of the venting cannula of FIG. 5.

FIG. 5B-5C illustrates schematically electrical venting valve options of the rectangular-shaped filter device of FIG. 5.

FIGS. 5D-5I illustrates schematically mechanical venting valve options of the rectangular-shaped filter device of FIG. 5.

FIG. 6 illustrates schematically an example suction leak device attached to a venting cannula.

FIG. 7A illustrates schematically an example manual pump leak device attached to a venting cannula.

FIG. 7B illustrates schematically the manual pump leak device of FIG. 7A being activated.

FIG. 7C illustrates schematically a cross-section along the axis 7C-7C of the manual pump leak device of FIG. 7A.

FIGS. 7D-F illustrate schematically activation of the example manual pump of FIG. 7A.

FIG. 8 illustrates schematically an example medical gases delivery cannula system with a leak device attached to a venting cannula on a patient.

FIG. 8A illustrates schematically a front view of the venting cannula of FIG. 8.

FIG. 8B illustrates schematically a cross-sectional view of the leak device of FIG. 8.

FIG. 8C illustrates schematically filter options of the leak device of FIG. 8.

FIG. 9A illustrates schematically an example sleeve leak device attached to an insufflation cannula.

FIG. 9B illustrates schematically a horizontal cross-section of the sleeve leak device of FIG. 9A.

FIG. 9C illustrates schematically a vertical cross-section of the sleeve leak device and the insufflation cannula of FIG. 9A with natural venting valve options.

FIG. 9D illustrates schematically a vertical-cross section of the sleeve leak device and the insufflation cannula of FIG. 9A with an electrical venting valve options.

FIG. 9E illustrates schematically a horizontal cross section of the sleeve leak device of FIG. 9D.

FIG. 9F illustrates schematically a close-up view of an example electrical valve of the sleeve leak device of FIG. 9D.

FIG. 9G illustrates schematically a vertical cross section of the sleeve leak device and the insufflation cannula of FIG. 9A with a heating element in the sleeve vent.

FIG. 9H illustrates schematically a horizontal cross section of the sleeve leak device of FIG. 9G.

FIGS. 9I-9M illustrate schematically additional attachment mechanisms for attaching the sleeve leak device of FIG. 9B to the cannula.

FIG. 10 illustrates schematically an example seal leak device attached to an insufflation cannula.

FIG. 11A illustrates schematically an example sleeve cap leak device attached to an insufflation cannula.

FIG. 11B illustrates schematically a cross-sectional view of the sleeve cap leak device attached to the insufflation cannula of FIG. 11A.

FIG. 12 illustrates schematically an example medical gases delivery cannula system with a cannula shaft sleeve attached to an insufflation cannula on a patient.

FIG. 12A illustrates schematically a front view of the cannula system of FIG. 12.

FIG. 12B illustrates schematically a front view of the cannula shaft sleeve of FIG. 12.

FIG. 12C illustrates schematically a vertical cross-sectional view of the cannula shaft sleeve of FIG. 12.

FIGS. 12D-12E illustrate schematically venting options of the cannula shaft sleeve of FIG. 12.

FIGS. 12F-12H illustrate schematically electrical connections in the cannula shaft sleeve of FIG. 12 to measure flow and control venting.

FIGS. 12I-12K illustrate schematically a heating element in the cannula shaft sleeve of FIG. 12 to reduce filter clogging.

FIGS. 12L-12O illustrate schematically securing attachment options of the cannula shaft sleeve of FIG. 12 to a cannula.

FIGS. 13A-13C illustrate schematically a capillary version of the cannula shaft sleeve of FIG. 12.

FIG. 13D-13G illustrate schematically securing attachment options of the capillary version of FIG. 13A to a cannula.

FIG. 14 illustrates schematically an example medical gases delivery cannula system with an insertable filter device attached to an insufflation cannula on a patient.

FIG. 14A illustrates schematically a front view of the cannula system of FIG. 14.

FIG. 14B illustrates schematically a front view of the insertable filter device of FIG. 14.

FIG. 14C illustrates schematically a vertical cross-sectional view of the insertable filter device of FIG. 14.

FIGS. 14D-14F illustrate schematically electrical connections in the insertable filter device of FIG. 14 to measure flow and control venting.

FIGS. 14G-14I illustrate schematically a heating element in the insertable filter device of FIG. 14 to reduce filter clogging.

FIGS. 14J-14M illustrate schematically securing attachment options of the insertable filter device of FIG. 14 to a cannula.

FIG. 14N-14Q illustrate schematically non-obstructing options of the insertable filter device of FIG. 14 to allow gases to flow through a cannula.

FIG. 15 illustrates schematically an example medical gases delivery cannula system with a hinged leak device attached to a venting cannula on a patient.

FIG. 15A illustrates schematically a front view of the venting cannula with the hinged leak device of FIG. 15.

FIG. 15B illustrates schematically a horizontal cross-sectional view of the venting cannula with the hinged leak device of FIG. 15.

FIG. 15C illustrates schematically a vertical cross-sectional view of a proximal portion of the venting cannula with the hinged leak device of FIG. 15.

FIGS. 15D-15F illustrate schematically electrical connections in the hinged leak device of FIG. 15 to measure flow and control venting.

FIGS. 15G-15I illustrate schematically a heating element of the hinged leak device of FIG. 15 to reduce filter clogging.

FIGS. 15J-15N illustrate schematically securing attachment options of the hinged leak device of FIG. 15 to a cannula.

FIGS. 16A-16B illustrate schematically an example separate venting port to a cannula.

FIGS. 17-23 illustrate schematically combinations of venting options disclosed herein.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

### Example Medical Gases Delivery Systems

Gases can be introduced to a surgical cavity, for example, the peritoneal cavity via a cannula inserted through an incision made in patient's body (for example, the abdominal wall). The cannula can be coupled to an insufflator. The gases flow from the insufflator can be increased to inflate the surgical cavity (for example, to maintain a pneumoperitoneum, which is a cavity filled with gas within the abdomen). The introduced gases can inflate the surgical cavity. A medical instrument can be inserted through the cannula into the inflated surgical cavity. For example, an endoscope, another scope, or camera unit can be inserted into the cavity and visibility in the cavity can be assisted by insertion of gases, which can be air or carbon dioxide. After initial insufflation and insertion of the instrument (for example, a laparoscope) through the primary cannula, additional cannulas can be placed in the surgical cavity under laparoscopic observation. Gases and/or surgical smoke can be vented from the surgical cavity using a venting attachment on one of the cannulas placed in the surgical cavity. At the end of the operating procedure, all instruments and cannulas are removed from the surgical cavity, the gases are expelled, and each incision is closed. For thoracoscopy, colonoscopy, sigmoidoscopy, gastroscopy, bronchoscopy, and/or others, the same or substantially similar procedure for introducing gases to a surgical cavity can be followed. The quantity and flow of gases can be controlled by the clinician performing the examination and/or automatically by the surgical system. The surgical system can be an insufflation system. The insufflator may deliver intermittent or continuous flow. The insufflator can control flow to ensure that the pressure in the surgical cavity is maintained at or around a predetermined range. The pressure allows for the surgical cavity to be inflated to a predetermined amount.

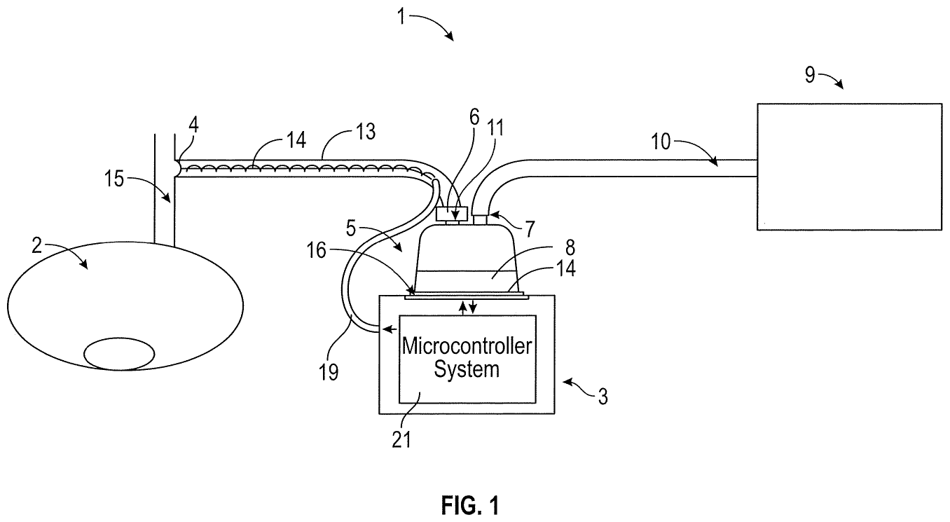

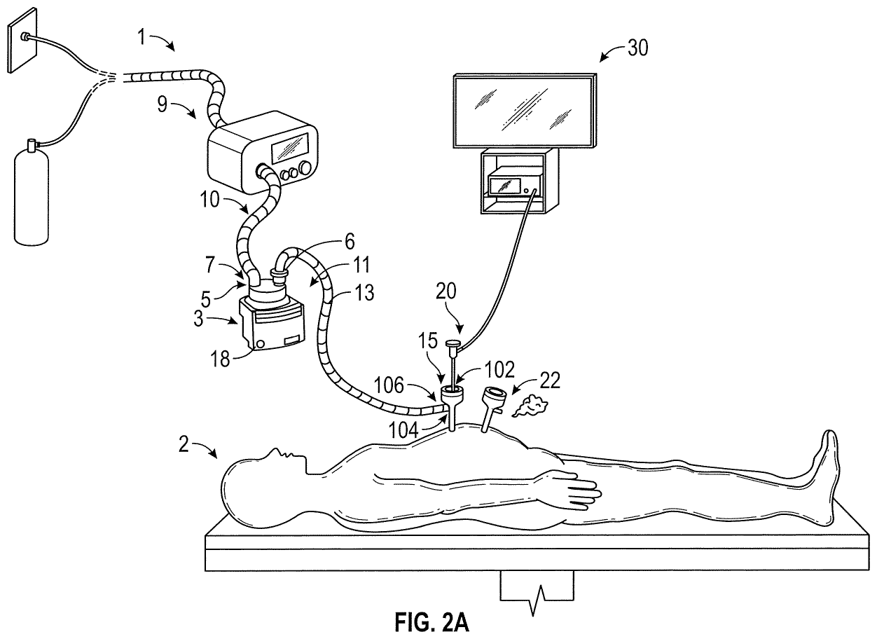

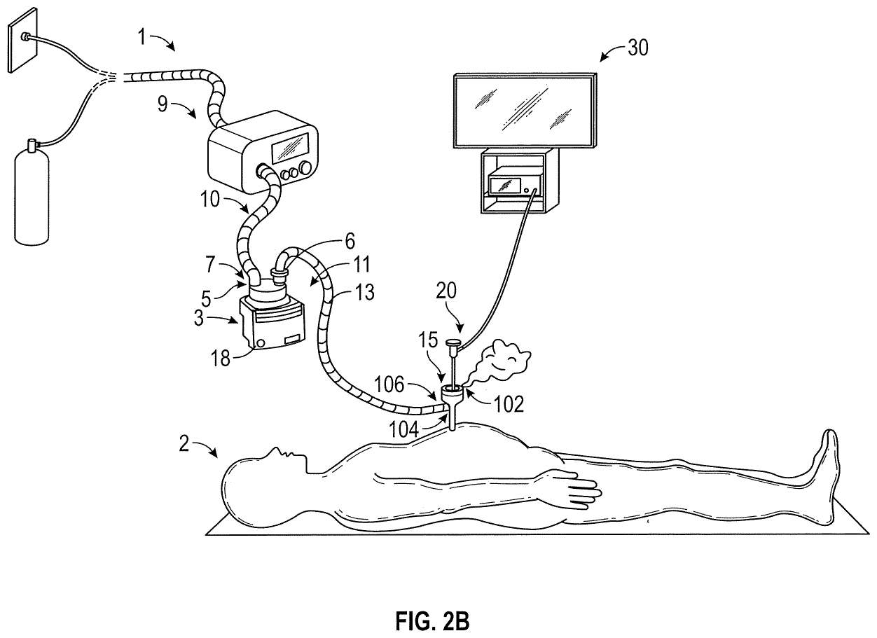

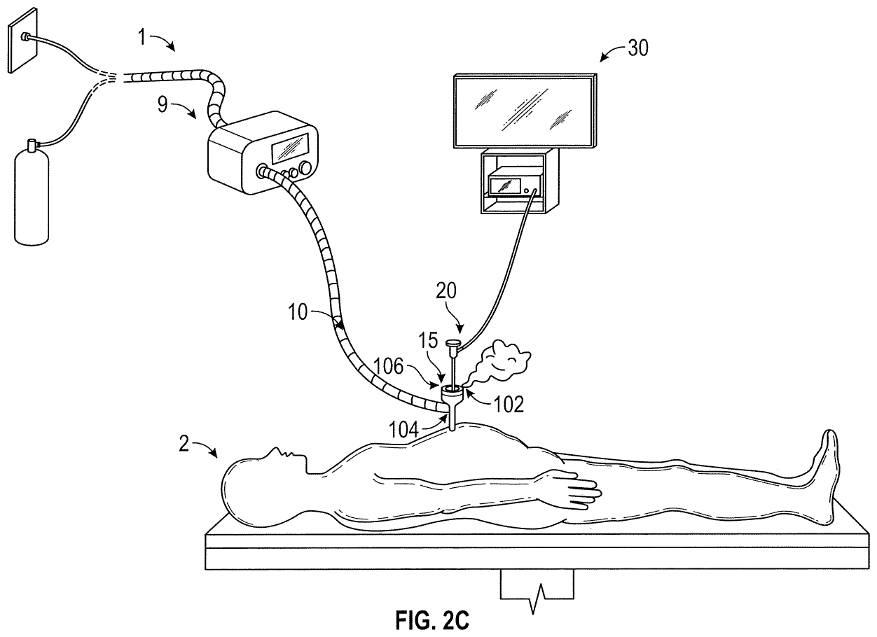

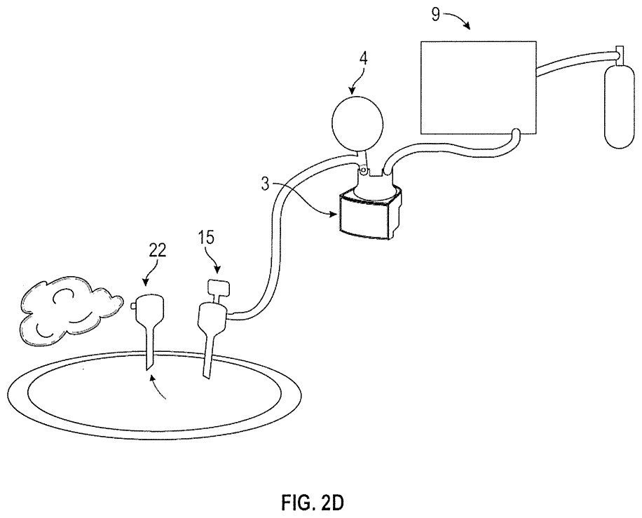

FIGS. 1 and 2A-D illustrate schematically using an example surgical system 1 during a medical procedure. Features of FIGS. 1 and 2A-D can be incorporated into each other. The same features have the same reference numerals in FIGS. 1 and 2A-D. As shown in FIG. 1, the patient 2 can have a cannula 15 inserted within a cavity of the patient 2 (for example, an abdomen of the patient 2 in the case of a laparoscopic surgery), as previously described. The cannula can be single use (disposable) or reusable. Alternatively, parts of the cannula can be single use (disposable) or reusable. The cannula may be made of materials that are biocompatible and/or sterilizable.

As shown in FIGS. 1 and 2A-D, the cannula 15 can be connected to a gases delivery conduit 13 (for example, via a Luer lock connector 4). The cannula 15 can be used to deliver gases into a surgical site, for example, within the cavity of the patient 2. The cannula 15 can include one or more passages to introduce gases and/or one or more medical instruments 20 into the surgical cavity. The medical instruments may be surgical instruments. The medical instrument can be a scope, electrocautery tool, or any other instrument. The medical instrument 20 can be coupled to an imaging device 30, which can have a screen. The imaging device 30 can be part of a surgical system which can include a plurality of surgical tools and/or apparatuses. The surgical system may be a surgical stack. In some configurations, the cannula 15 can be used in a system that includes a supplementary gases source.

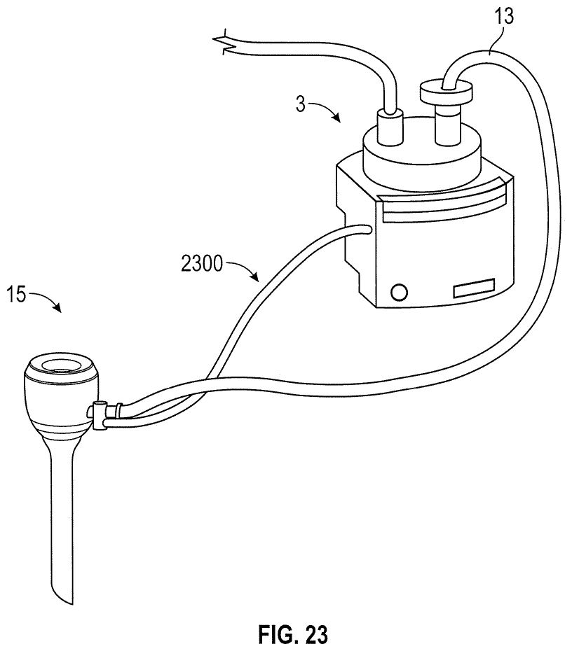

As shown in FIGS. 2A and 2D, the system can include a venting cannula 22, which can have substantially the same features as the cannula 15. The venting cannula may include a leak device coupled to the venting cannula. The leak device may include a valve that allows and/or controls venting. Alternatively the leak device may have passive venting structures. The leak device may also be shaped or may include features that control the venting rate out of the surgical cavity. The valve can be automatically controlled by a controller associated with the gases source (e.g., insufflator) or by a controller in the humidifier or an external controller. The valve can also be manually actuated (for example, by turning a tap by hand or by a foot pedal, or otherwise). The leak device can include a filtration system to filter out smoke and the like. The venting cannula 22 can also alternatively be coupled to a recirculation system (see FIG. 23) that is configured to recirculate the gases from the surgical cavity back to the insufflator for re-delivery into the surgical cavity. The gases can also be filtered and/or dehumidified prior to being returned to the insufflator. As shown in FIGS. 2B and 2C, the cannula 15 can include a venting attachment so that a venting cannula 22 may not be necessary. The cannula 15 may include two or more passages. One passage can be configured to deliver gases and/or the medical instrument into the surgical cavity. Another passage can be configured to vent gases out of the surgical cavity.

As shown in FIGS. 1, 2A, 2B, and 2D, the gases delivery conduit 13 can be made of a flexible plastic and can be connected to a humidifier chamber 5. The humidifier chamber 5 can optionally or preferably be in serial connection to an insufflation fluid (for example, insufflation gases) supply 9 via a further conduit 10. The insufflation fluid supply, or insufflation fluid source can be, for example, an insufflator, bottled gases, or a wall gases source. The insufflation fluid supply 9 can provide the gases without humidification and/or heating. As shown in FIG. 2D, an accumulator 4 can be connected downstream of the humidifier's outlet 11. The accumulator 4 can provide a continuous flow of gases by having a substantially constant leak rate to the cannula 15. A filter 6 be connected downstream of the humidifier's outlet 11. The filter can also be located along the further conduit 10, or at an inlet of the cannula 15. The filter can be configured to filter out pathogens and particulate matter in order to reduce infection or contamination of the surgical site from the humidifier or gases source. The insufflation fluid supply can provide a continuous or intermittent flow of gases. The further conduit 10 can also preferably be made of flexible plastic tubing. The insufflation fluid supply 9 can provide, for example, dry cold fluid, dry hot fluid, or humidified fluid.

The insufflation fluid supply 9 can provide one or more insufflation fluid, for example, carbon dioxide, to the humidifier chamber 5. The gases can be humidified as they are passed through the humidifier chamber 5, which can contain a volume of humidification fluid 8, such as water for example.

A humidifier that incorporates the humidifier chamber 5 can be any type of humidifier. The humidifier chamber 5 can include a plastic formed chamber having a metal or otherwise conductive base 14 sealed thereto. The base can be in contact with the heater plate 16 during use. The volume of humidification fluid 8 contained in the chamber 5 can be heated by a heater plate 16, which can be under the control of a controller or control means 21 of the humidifier. The volume of humidification fluid 8 within the chamber 5 can be heated such that it evaporates, mixing humidification fluid vapor with the insufflation fluid flowing through the chamber 5 to heat and humidify the insufflation fluid. The humidification fluid may be water.

The controller or control means 21 can be housed in a humidifier base unit 3, which can also house the heater plate 16. The heater plate 16 can have an electric heating element therein or in thermal contact therewith. One or more insulation layers can be located between in the heater plate 16 and the heater element. The heater element can be a base element (or a former) with a wire wound around the base element. The wire can be a nichrome wire (or a nickel-chrome wire). The heater element can also include a multi-layer substrate with heating tracks electrodeposited thereon or etched therein. The controller or control means 21 can include electronic circuitry, which can include a microprocessor for controlling the supply of energy to the heating element. The humidifier base unit 3 and/or the heater plate 16 can be removably engageable with the humidifier chamber 5. The humidifier chamber 5 can also alternatively or additionally include an integral heater. The controller or control means can be housed in the insufflator, the cannula, the humidifier, and/or be external to the aforementioned components.

The heater plate 16 can include a temperature sensor, for example, a temperature transducer or otherwise, which can be in electrical connection with the controller 21. The heater plate temperature sensor can be located within the humidifier base unit 3. The controller 21 can monitor the temperature of the heater plate 16, which can approximate a temperature of the humidification fluid 8. The humidification fluid may be water.

A temperature sensor can also be located at the or near the outlet 11 to monitor a temperature of the humidified gases leaving the humidifier chamber 5 from the outlet 11. The temperature sensor can also be connected to the controller 21 (for example, with a cable or wirelessly). Additional sensors can also optionally be incorporated, for example, for sensing characteristics of the gases (for example, temperature, humidity, flow, or others) at a patient end of the gases delivery conduit 13.

The gases can exit out through the humidifier's outlet 11 and into the gases delivery conduit 13. The gases can move through the gases delivery conduit 13 into the surgical cavity of the patient 2 via the cannula 15, thereby inflating and maintaining the pressure within the cavity. Preferably, the gases leaving the outlet 11 of the humidifier chamber 5 can have a relative humidity of, for example, up to around 100%, for example at 100%. As the gases travel along the gases delivery conduit 13, further condensation can occur so that humidification fluid vapor (water vapor, for example) can condense on a wall of the gases delivery conduit 13. Further condensation can have undesirable effects, for example, detrimentally reducing the humidification fluid content of the gases delivered to the patient. In order to reduce and/or minimize or prevent the occurrence of condensation within the gases delivery conduit 13, a heating element, such as, for example, a heater wire 14 can be provided within, throughout, or around the gases delivery conduit 13. The heater wire 14 can be electronically connected to the humidifier base unit 3, for example by an electrical cable 19 to power the heater wire. In some embodiments, other heating elements could be included in addition or alternatively, e.g., a conductive ink, conductive polymers, or a flexible PCB. In some embodiments, other heating elements could be included in addition or alternatively. In some cases, the PCB could be flexible, or rigid and pre-shaped to an arcuate shape for example. In some embodiments, the heating element could be, for example, discrete Positive Temperature Coefficient (“PTC”) heaters, or heaters including conductive plastic/polymer. Optionally, the heating element can include an inductive heating element. Optionally, the heating element can include a chemical heating element, for example, silica beads. Optionally, the cannula can be pre-heated prior to insertion.

The heater wire 14 can include an insulated copper alloy resistance wire, other types of resistance wire, or other heater element, and/or be made of any other appropriate material. The heater wire can be a straight wire or a helically wound element. An electrical circuit including the heater wire 14 can be located within walls of the gases delivery tube 13. The gases delivery tube 13 can be a spiral wound tube. Alternatively, the gases delivery tube 13 can include a non-helical or straight tube. Optionally, the gases delivery tube 13 can be corrugated or non-corrugated. The heater wire 14 can be spirally wound around an insulating core of the gases delivery conduit 13. The insulating coating around the heater wire 14 can include a thermoplastics material which, when heated to a predetermined temperature, can enter a state in which its shape can be altered and the new shape can be substantially elastically retained upon cooling. The heater wire 14 can be wound in a single or double helix. Measurements by the temperature sensor and/or the additional sensor(s) at the patient end of the conduit 13 can provide feedback to the controller 21 so that the controller 21 can optionally energize the heater wire to increases and/or maintain the temperature of the gases within the gases delivery conduit 13 (for example, between approximately 35° C. and 45° C.) so that the gases delivered to the patient can be at or close to 37° C. or any other suitable temperature, including but not limited to temperatures above or below the internal body temperature (for example, approximately 5, 10, or 15 degrees above or below 37° C.). Alternatively or additionally the system can include additional sensors configured to measure one or more parameters, e.g., ambient temperature and ambient humidity sensors; and/or flow sensors, and/or pressure sensors configured to determine flow rate or pressure of flow or determine the pressure within a cavity or in the tube. Additionally or alternatively the system may also include additional sensors. The sensors can be located upstream, downstream, and/or within the humidifier. The sensors may be configured to determine a parameter of the insufflation fluid or one or more parameters of the patient/surgical cavity. Each of the sensors can provide feedback information to one or more controllers which in turn can provide closed loop feedback to keep humidity, temperature, flow, pressure, or other parameters within desired parameters, e.g., a preset range.

The controller or control means 21 can, for example, include the microprocessor or logic circuit with associated memory or storage means, which can hold a software program. When executed by the control means 21, the software can control the operation of the surgical system 1 in accordance with instructions set in the software and/or in response to external inputs. The surgical system can be an insufflation system. For example, the controller or control means 21 can be provided with input from the heater plate 16 so that the controller or control means 21 can be provided with information on the temperature and/or power usage of the heater plate 16. The controller or control means 21 can be provided with inputs of temperature of the gases flow. For example, the temperature sensor can provide input to indicate the temperature of the humidified gases flow as the gases leave the outlet 11 of the humidifier chamber 5. A flow sensor can also be provided in the same position as or near the temperature sensor or at other appropriate location within the surgical system 1. The controller 21 can control a flow regulator which regulates the flow rate of gases through the system 1. The regulator can include a flow inducer and/or inhibiter for example, a motorized fan or pump. Valves and/or vents can additionally or alternatively be used to control the gases flow rate.

A patient input 18 located on the humidifier base unit 3 can allow a user (for example, a surgeon or nurse) to set a desired gases temperature and/or gases humidity level to be delivered. Other functions can also optionally be controlled by the user input 18, for example, control of the heating delivered by the heater wire 14. The controller 21 can control the system 1, and in particular to control the flow rate, temperature, and/or humidity of gas delivered to the patient, to be appropriate for the type of medical procedure for which the system 1 is being used.

The humidifier base unit 3 can also include a display for displaying to the user the characteristics of the gas flow being delivered to the patient 2.

Although not shown, the humidifier can also optionally be a passover or bypass humidifier, which can include the chamber with a volume of water or any other type of humidification fluid, but may not include a heater plate for heating the humidification fluid (for example, water). The chamber can be in fluid communication with the insufflation fluid supply such that the insufflation fluid(s) are humidified by the humidification fluid vapor (for example, water vapor) evaporated from the volume of humidification fluid as the insufflation fluid(s) pass over the volume of humidification fluid.

When in use, the humidifiers described above can be located outside an “operating sterile zone” and/or adjacent the insufflator. As a result, the medical personnel would not be required to touch the humidifier when moving the cannula during the operation to maneuver the medical instruments within the surgical cavity. The humidifier may not need to be sterilized to the same extent as the medical instruments. Furthermore, the humidifier being located outside the “operating sterile zone” can reduce obstructions to the medical personnel during the operating procedure that may restrict movements of the medical personnel and/or the medical instruments in the already crowded space.

As shown in FIG. 2C, the system may be used without a humidifier so that the insufflation fluid supply 9 can be coupled directly to the cannula 15. The humidifier can be an optional unit. Humidifying the insufflation fluid reduces cellular damage or desiccation.

### Examples of Venting Attachments or Leak Devices

The present disclosure provides examples of a venting attachment or leak device configured to vent insufflation fluid and/or surgical smoke from the surgical cavity. The venting attachment or leak device may be connected to either the cannula 15 or cannula 22. The venting attachment or leak device may be connected to a cannula initially used for insufflation and the delivery of insufflation fluid can be subsequently performed by another cannula. The venting attachment can improve optical clarity and/or maintain a clearer field of vision (for example, by clearing camera lens fog, condensation and/or smoke), reduce and/or minimize surgical smoke caused by electrosurgery, electrocautery, energy and laser cutting and/or cauterizing, among others, aid in maintaining a substantially stable operating space within the surgical cavity (for example, by maintaining a substantially stable insufflation fluid pressure), and/or reduce operating time and/or post-operative complications (for example, pain and/or other side effects). In the present disclosure, features of the different examples of venting attachment or leak device can be incorporated into or combined with one another.

During these operations/procedures there is often also a smoke plume that is generated. The smoke plume can engulf the scope or move across the scope and restrict vision of the surgeon. Clearing the smoke as well as clearing the smoke plumes helps in improving optical clarity during surgery. This makes surgery safer, faster and more efficient. A high concentration of smoke in the insufflated cavity, and in the field of vision can severely impede the optical clarity when viewing the space inside the peritoneum or other site through a vision system, including but not limited to, a scope or a camera unit, inserted through a trocar. A trocar includes a cannula and an obturator. Without the use of venting or suction, surgeons generally have no option but to release all the gas from inside the pneumoperitoneum through deflation, then re-insufflation. The leak rate can be configured to balance the need to clear surgical smoke and the need to maintain substantial stability of the surgical cavity. The venting attachment and/or leak device can be dimensioned such that the leak device has a set venting flow rate. The venting flow rate may be greater than or equal to the delivery flow rate. A venting rate greater than or equal to a gases delivery flow rate can prevent the surgical cavity from over-inflating or over-pressuring and/or causing damage. In some configurations, the venting device can have a leak rate that does not exceed the delivery flow rate. The venting device can include structures that control the venting flow rate such that the venting flow rate is less than or equal to the delivery flow rate. A venting rate equal to or less than the gases delivery flow rate can assist in maintaining a more stable pressure in the surgical cavity. Preferably, the venting rate is equal to the gases delivery flow rate so as to maintain a more stable surgical cavity. The venting device can be configured such that it does not negatively affect the surgical cavity stability. In a configuration where the venting flow rate is greater than the gases delivery flow rate, the venting flow rate is limited such that it does not deflate the surgical cavity.

The venting attachment or leak device can also include one, two, or more filters configured to remove potentially hazardous chemicals and/or particles before releasing the gases and/or smoke into the atmosphere. The filter can be configured to filter particles as small as, for example, about 0.1 microns to about 0.2 microns, or about 0.12 microns. The filter can be configured to filter the particles with at least about 99% efficiency, or about 99.999% efficiency, or about 99.9995% efficiency. The filter can be an ultra-low particulate air (ULPA) filter. The filter can also include optionally a carbon filter to reduce odor. The filters could also be a high-efficiency particulate air (HEPA) filter. The filters can include multiple filter elements that can be positioned in series. For example, ULPA filters and carbon filters can be positioned in series. The filters throughout the disclosure can also include a filter housing which has features to reduce or absorb humidity or condensation in the filters, for example, with water (or other humidification fluid) traps, a desiccant, and/or the like.

The example venting attachments or leak devices can be attached to the insufflation cannula 15 or the venting cannula 22, or be used in a standalone manner. The cannula 15, 22 can have an upper housing 102 connected to an elongate shaft 104. The elongate shaft 104 can optionally have a square tip or a pointed end such that the cannula can function as a trocar for easier insertion of the cannula 100 into the surgical cavity. The upper housing 102 can have a greater cross-sectional dimension than the elongate shaft 104 for easier insertion of the medical instruments. As shown in FIGS. 2A-2C, the upper housing 102 can have generally a funnel shape, with a cross-sectional dimension (for example, diameter) decreasing from a location further from the elongate shaft 104 to a location closer to the elongate shaft 104. A gases inlet or outlet, or gases port 106 can be located on the upper housing 102. The upper housing 102 can include an opening. Standard cannula seals can be used to seal the opening. The seals can have slits to allow insertion of a medical instrument. The elongate shaft 104 can include a lumen. The opening can be in fluidic communication with the shaft lumen. The venting attachment or leak device can be coupled to the gases inlet or outlet 106, along the elongate shaft 104, and/or to the upper housing 102. Cannulas 22 for example, described above can be utilized as or modified for use, or including attachments to create a venting cannula. In some embodiments, a cannula, or attachment to a cannula with venting passages and gas delivery passages can be advantageous because it provides a single device that can be used to deliver gases and vent out smoke and other gases from the surgical cavity, thereby helping to maintain a clear field of vision.

In some embodiments, a surgical cannula includes a housing, an elongate shaft extending from the housing, and at least one lumen within the elongate shaft. The cannula can include gripping features to grip against the surgical cavity. The housing can include one or more seals/valves disposed adjacent an instrument opening. The seals/valves are configured to seal against an instrument inserted through the instrument opening. A gases inlet is disposed on the housing or the shaft. The gases inlet can be in fluid communication with the lumen and receives gases from either a humidifier or an insufflator. The surgical cannula may include other features for example, external seals and may include either a flat (e.g., square) tip or an angled (e.g., beveled) tip. Any number of venting features as disclosed elsewhere herein can be incorporated into, or attached to cannulas as described.

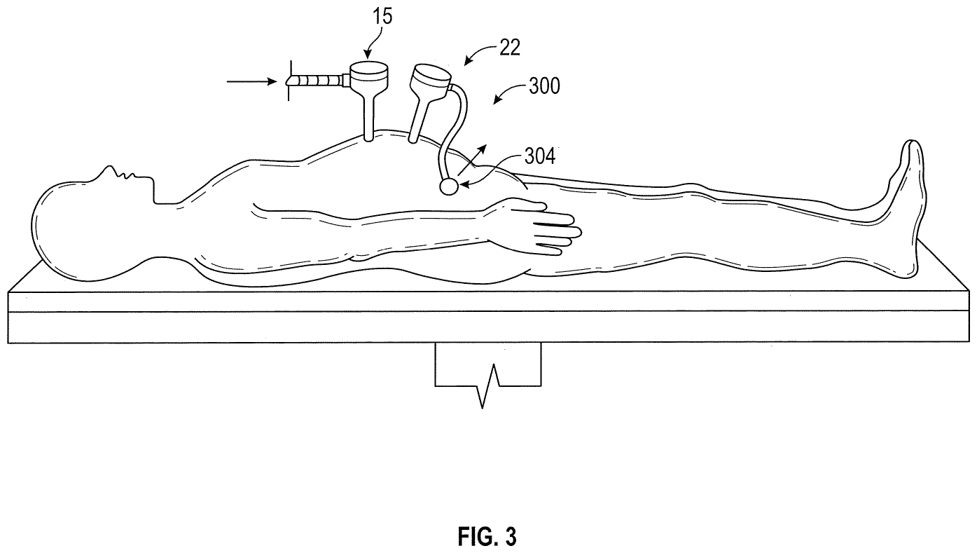

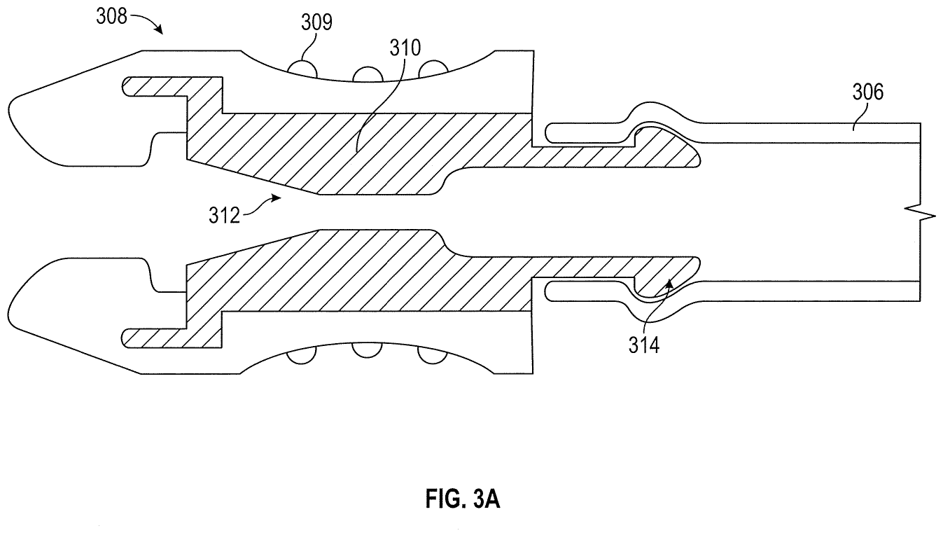

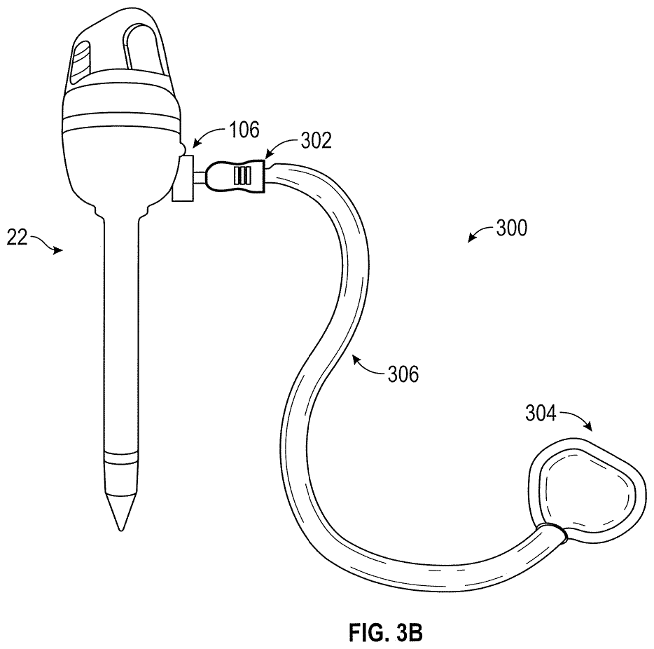

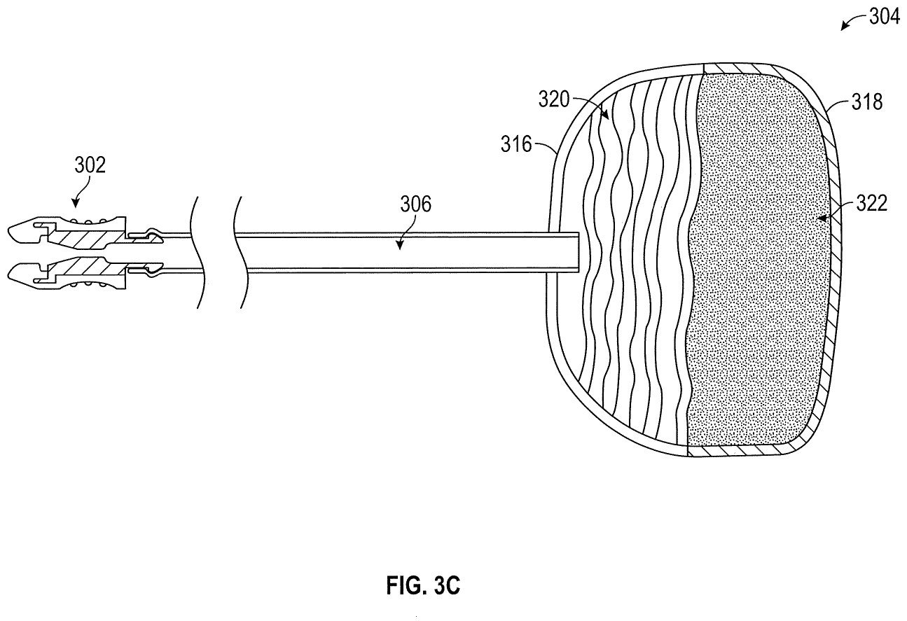

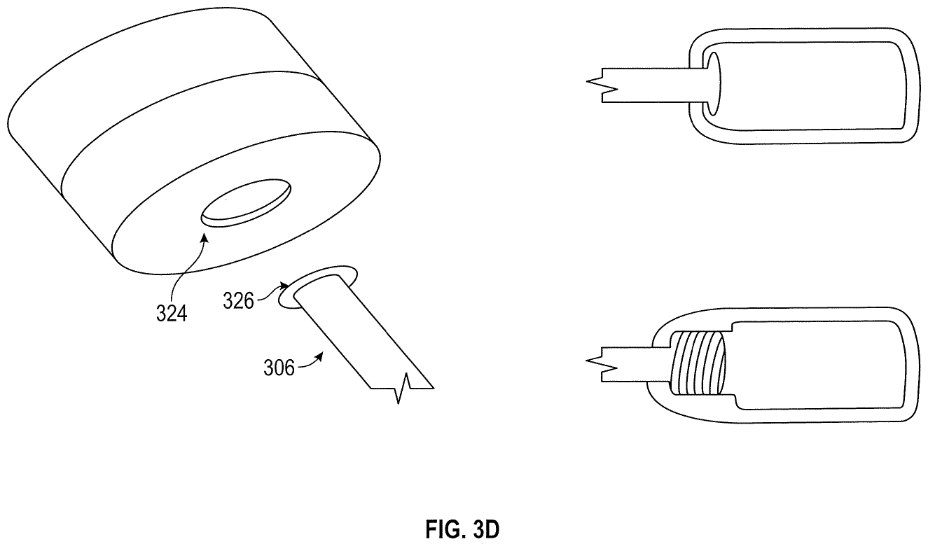

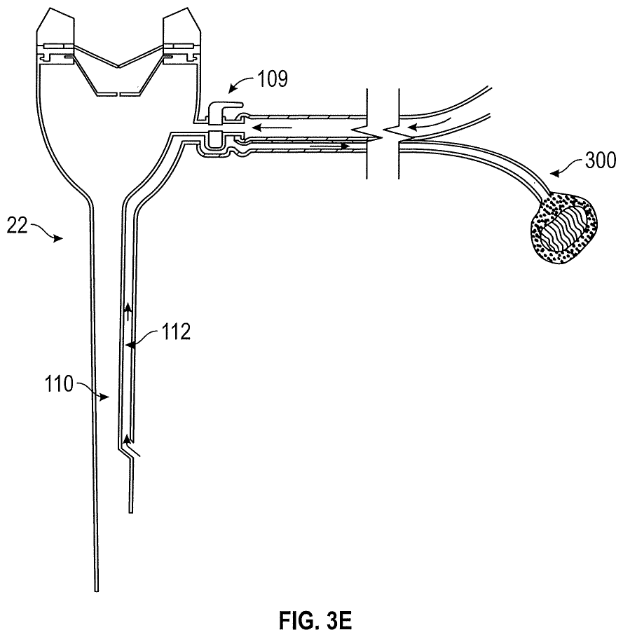

More detailed examples of the leak devices are described below with reference to FIGS. 3-23. Reference numerals of the same or substantially the same features share the same last two digits.

Examples of a Leak Device Attached to the Gases Port of a Cannula

FIGS. 3-3E illustrate examples of a suspended filter device 300 configured to be coupled to a venting cannula 22. As shown in FIG. 3, the venting cannula 22 can be a different cannula than the insufflation cannula 15 configured to deliver insufflation fluid into and/or allow a medical instrument to be inserted into a surgical cavity within a patient's body.

As shown in FIG. 3A, a venting flow control device 308 can be connected to the gases port 106 of the cannula 22. The venting flow control device 308 is designed to control the leak rate, that is, the venting flow rate out of the surgical cavity. The leak device is designed to include a flow restriction that controls the venting flow rate such that the pressure within the surgical cavity is maintained within predetermined limits.

The device 308 can include hand grip features 309 for ease of use when pushing or pulling the device 308. The venting flow control device 308 can include a resilient material, for example, rubber, elastomer or the like. The resilient material of the venting flow control device 308 can extend around a more rigid (for example, made of harder plastic) barb connector 310. For example, the resilient material of the venting flow control device 308 can be overmoulded onto the barb connector 310. The venting flow control device 308 can include a tapering 312 from a proximal opening of the venting flow control device 308 with a size of the opening decreasing toward a proximal end of barb connector 310. The venting flow control device 308 can include a flow restriction in a passage of the barb connector 310. The resilient material can encase the flow restriction and the barb connector 310. The flow restriction is shaped and dimensioned to define a venting flow rate, for example, to control the venting flow. The venting flow rate may be greater than or equal to the delivery flow rate. The venting flow rate can also be less than or equal to the delivery flow rate to prevent deflation of the surgical cavity. The tapering or restriction in the opening of the venting flow control device 308 can also aid in controlling a flow rate of the gases and/or smoke passing into a filter (see FIG. 3C) of the leak device 300. An elongate tube 306 can be connected to the barb 314 of the barb connector 310 by a push fit or overmoulded onto the barb 314 to secure the tube 306 to the venting flow control device 308. The barb connector 310 may be configured to connect to a Luer connector on the cannula. The resilient material overmould can allow the connecting end of the venting flow control device 308 to flex and conform to a connector, for example, a Luer connector. The Luer connectors disclosed herein do not restrict the venting path. The Luer connectors in the venting attachment examples disclosed herein can have a lumen configured to allow a venting rate of, for example, about 1-10 L/min, or about 5-7 L/min, or about 4 L/min. The Luer connector may be made from a flexible material, (for example, elastomers or rubber) that can conform around the gases port to seal around the gases port. The Luer connector can include an opening, a neck region, and/or a confined area. The neck region can be immediately adjacent the opening and the confined area. The neck region can deform to allow passage of the gases port of the cannula, for example, of a flanged end portion of the gases port. The confined area can receive and retain the flanged end portion of the gases port. The neck region can conform around an outer surface of the gases port, for example, along a shaft portion of the gases port, to form a seal. The venting flow control device 308 can be incorporated into any of the leak device examples coupled to the cannula gases port 106. The venting flow control device 308 can be coupled to any suspended leak device (see FIGS. 3C, 4B, 5B, and 8B-8C).

As shown in FIG. 3B, a suspended filter device 300 can include a cannula connecting end 302 and a venting end 304. The elongate flexible tube 306 can connect the cannula connecting end 302 and the venting end 304. The cannula connecting end 302 can be connected to the gases port 106 of the cannula 22. The cannula connecting end 302 may include a Luer connector. The cannula connecting end 302 may include the venting flow control device 308. As shown in FIG. 3, the venting end 304 can be located outside the surgical cavity and outside the patient's body. The venting end 304 can have a shape that increases in cross-sectional dimension distally with respect to proximally, for example, in a partial dome shape, and can be greater in a cross-sectional dimension than an outer diameter of the tube 306.

As shown in FIG. 3C, the venting end 304 can be enclosed by a gas impermeable lining 316 and a gas permeable membrane 318. A particulate filter (for example, an ULPA filter) 320 can be located within a portion of the venting end 304 lined by the gas impermeable lining 316, which can prevent gases from escaping through the lining 316. An odor filter (for example, a carbon filter) 322 can be located within another portion of the venting end 304 lined by the gas permeable membrane 318 so that the gases that have been filtered can exit through the membrane 322. In the venting end of the leak device examples disclosed herein, the vented gases can generally diffuse first through the particulate filter and then the odor filter before exiting a gas permeable membrane or vents. The arrangement and/or types of filters can be varied in some configurations.

When in use, insufflation fluid and/or surgical smoke within the surgical cavity can enter into the lumen in the elongate shaft 104 of the cannula 22 and travel into the device 300 via the gases port 106. The gases and/or smoke can diffuse through the porous fabric filter material within the venting end 304. The gases and/or smoke can exit the membrane 318 at the venting end 304 after having been filtered by the particulate filter 320 and also optionally the odor filter 322.

FIG. 3D illustrates an example Luer connection of the tube 306 to a replaceable filter bag, for example, the suspended filter of FIG. 3A or filter bag of other shaped disclosed herein, in the venting end 304. The replaceable filter bag can include a female Luer connector 324. A distal end of the tube 306 can include a male Luer connector 326. As shown in FIG. 3D, the male Luer connector 326 can be pushed into or spirally engage the female Luer connector 324. Other forms of filter bag attachment to the tube (for example, adhesives, friction, or others) can also be used.

The suspended filter device 300 can also be coupled to a gases port 109 of a double-lumen cannula 22. The gases port 109 can include a first entry in fluidic communication with an insufflation lumen 110 and a second entry in fluidic communication with an offset venting lumen 112. The device 300 can be coupled to the second entry, separate from the gases delivery tube, for example, the gases delivery conduit 13 describe above.

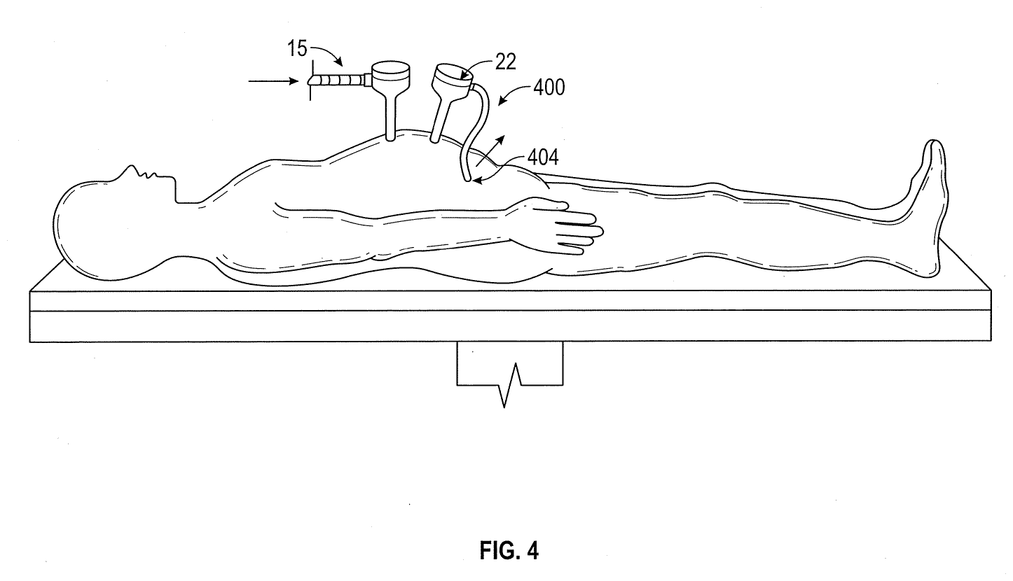

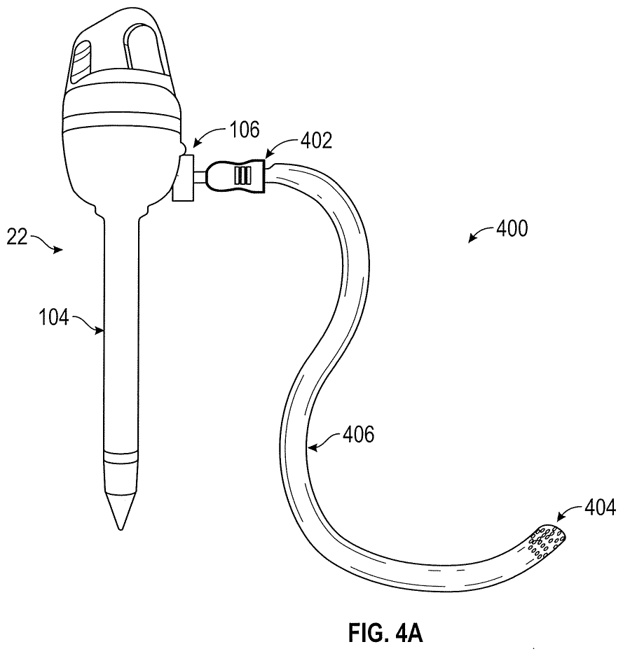

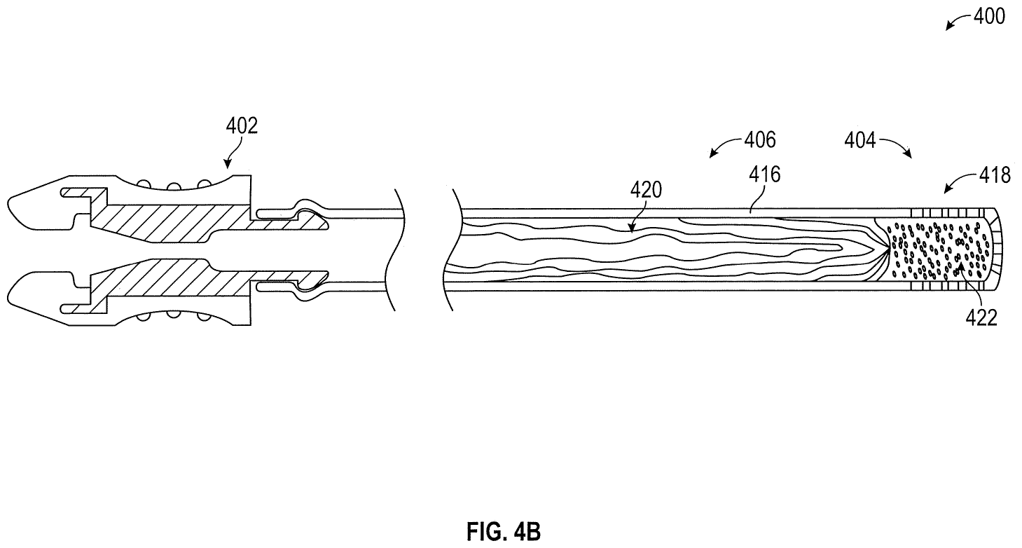

FIGS. 4 and 4A-4B illustrate another suspended filter device 400. As shown in FIGS. 4 and 4A-4B, the venting end 404 of the device 400 can have a similar cross-sectional dimension as the outer diameter of the tube 406 such that the device 400 has an elongate and flexible shape, including snake-like shapes. The device 400 can have any of the features of the suspended filter device 300.

As shown in FIG. 4B, the tube 406 can include a particulate filter (for example, an ULPA filter) 420 within at least a distal portion of the lumen of the tube 406. A gas impermeable flexible material (for example, a typical tube material) 416 can prevent the gases and/or smoke from escaping through the wall of the tube 406. The gases path within the lumen of the tube 406 can decrease (for example, gradually decrease) due to the surrounding particulate filter 420 toward a distal end of the tube 406. The venting end 404 can contain an odor filter 422 (for example, a carbon filter for example). The wall of the venting end 404 can include a plurality of vent holes 418 configured to allow the filtered gases and/or smoke to exit the venting end 404. In some configurations, the distal end of the tube 406 can optionally include an overmoulded threaded section threadedly engaging a corresponding threaded portion of the venting end 404.

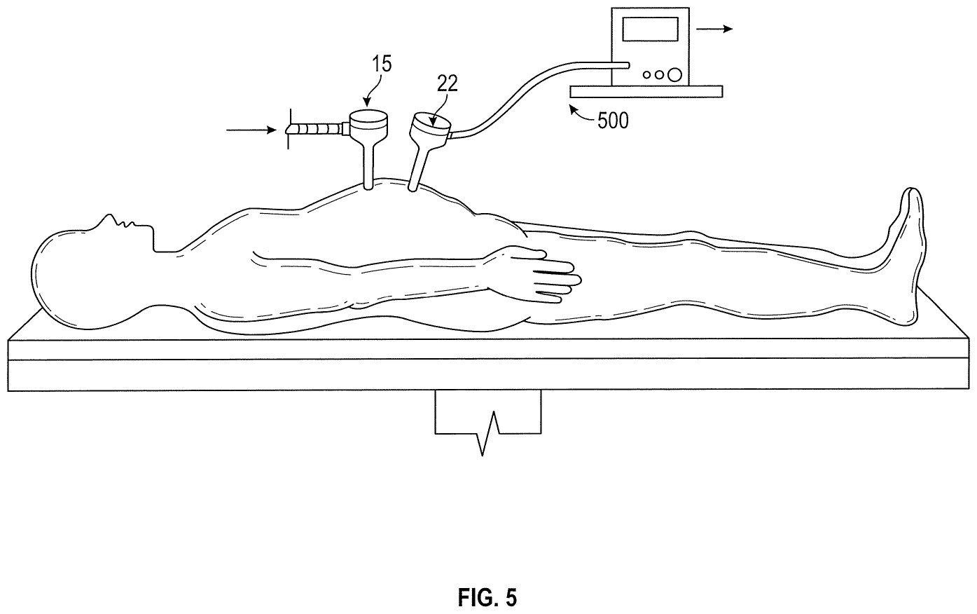

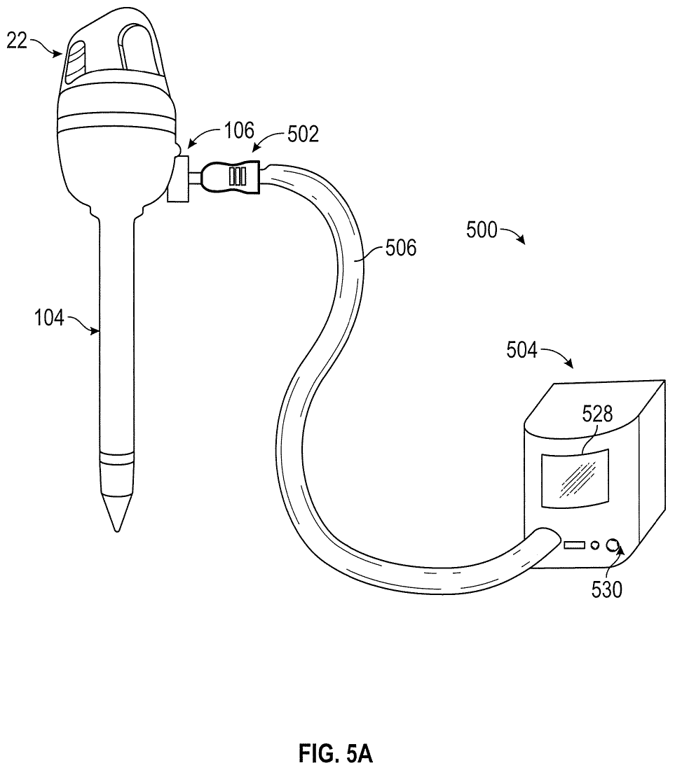

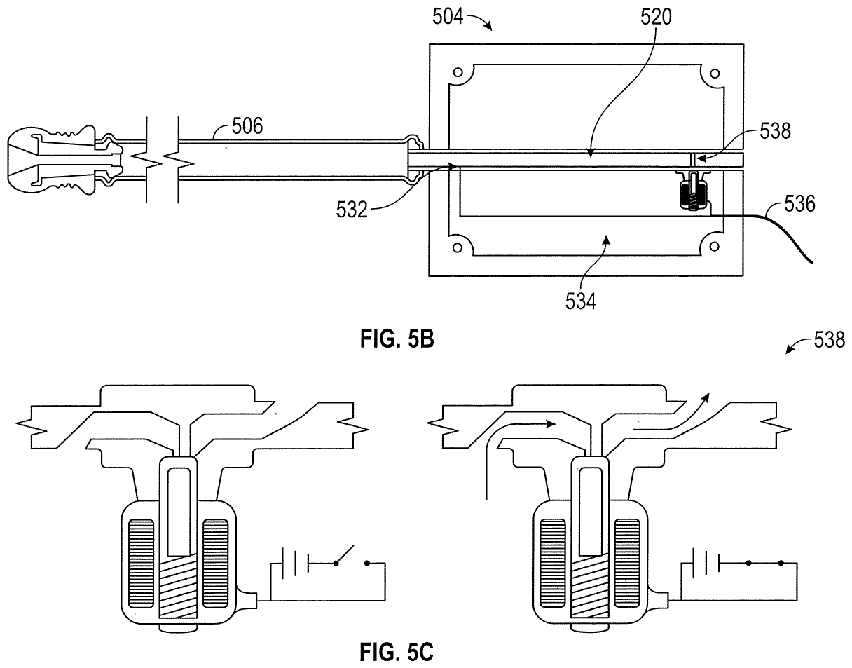

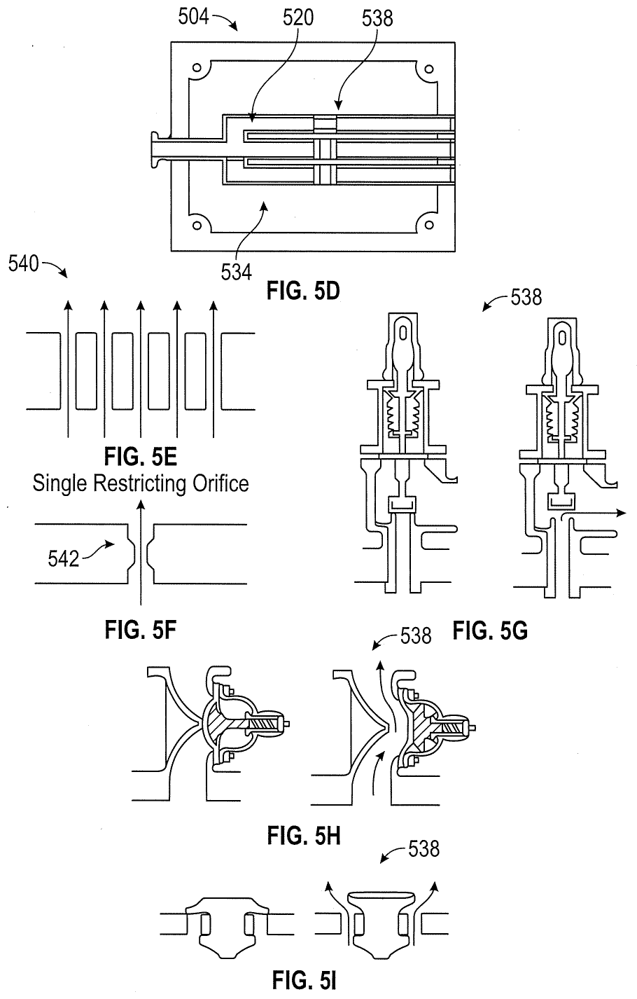

FIGS. 5 and 5-5I illustrate a filter device 500. The device 500 can have any of the features of the devices 300, 400. As shown in FIGS. 5 and 5A-5B, the venting end 504 of the device 500 can include a unit (such as a generally rectangular shaped unit, including box shapes). The venting end 504 can include a user display 528 so as to show a user the leak or venting rates of the gases, optionally a composition of the gases being vented, and/or other information. The vented gases composition can be an indication of the amount of surgical smoke removed from the surgical cavity. The venting end can optionally include user interface 530, which can control the venting rate.

As shown in FIG. 5B, the venting end 504 can include a gases venting path. The gases venting path and/or at least a portion of the tube 506 can include the particulate filter 520 (and optionally an odor filter). The gases venting path can include one or more sensors, for example, a flow and/or smoke sensor 532 configured to take measurements of the gases entering the venting end 504. The venting end 504 can include a printed circuit board (PCB) 534 in electrical communication with the sensor 532 and configured to process the readings from the sensor 532. The PCB 534 can also be configured to provide data for display on the user display 528. Power can be provided to the PCB by electrical communication (for example, with a power cord 536) to the mains.

As shown in FIG. 5B, the gases venting path can have a valve 538 controlling an opening of the gases venting path to the atmosphere. The valve 538 can be electrically controlled. As shown in FIG. 5C, the valve 538 can be a solenoid activated relief valve. The valve can be initially closed by a spring force, when the switch is open so that the electrical control circuit is turned off. When the switch is closed so that the electrical circuit forms a closed loop, the solenoid can be activated to open the valve.

In some embodiments, the one, two, or more sensors may provide feedback to a controller operably connected to a humidifier or an insufflation fluid supply (e.g. insufflator). The venting lumen may fluidly couple to a gas evacuation system e.g., a suction unit. The sensor outputs can control the gas evacuation system. Furthermore, the sensor output may control the output valves to control the venting rate in some embodiments.

As shown in FIG. 5D, the venting end 504 can also optionally include mechanical venting valves 538. The venting end 504 can include a plurality of (for example, two, three, or more) gases venting paths. As shown in FIGS. 5E and 5F, the openings can include a plurality of small orifices 540. Each orifice 540 can include a restricting neck 542. The venting end 504 can include a plurality of venting options (for example, of high flow, medium flow, and/or low flow). The mechanical venting valves 538 (for example, ball valve switches) can be located at an opening of the gases venting path. The valves 538 for different gases venting paths can be selectively opened or closed to adjust the overall venting rate.

As shown in FIG. 5G, the mechanical valve 538 can be a standard pressure relief valve. The valve 538 can be initially closed by a spring pressure. When the spring pressure is lower than an internal pressure within the gases venting path, the valve can be opened.

As shown in FIG. 5H, the mechanical valve 538 can be a diaphragm pressure relief valve. The valve 538 can be initially closed by a spring pressure. A flexible plastic seal of the valve 538 can pop open when a pressure greater than the spring pressure is applied by the flow of gases in the gases venting path.

As shown in FIG. 5I, the mechanical valve 538 can be an umbrella pressure relief valve. The valve 538 can initially snap close when no pressure is applied. When a pressure greater than a threshold pressure (for example, about 15 mmHg or any other value) is applied, the flexible seal (for example, made of rubber or elastomer) of the valve 538 can pop open.

In some configurations, valves can be configured in order to control the venting rate, e.g., the rate of gases being vented out of the cannula. The venting rate can be controlled to a predetermined rate such that smoke is cleared, that is, smoke plumes are cleared from the surgical cavity. The solenoid valves may be controlled by a controller, e.g. a controller in a humidifier, a controller in the insufflator, a controller in the cannula, or an external controller. A controller may be associated with both the gases source (e.g., insufflator) and the humidifier. The controller associated with the gases source and/or the humidifier may be external from the gases source and/or the humidifier. The controller may also be positioned internally within the cannula.

In some embodiments, a valve may be passive or active. Passive valves, e.g., spring valves or umbrella valves, are configured to vent at a predetermined pressure. The venting pressure corresponds to a pressure that can be used to maintain a constant pressure in the surgical cavity and provide a desired venting rate. Active valves are preferably actively controlled to achieve a constant pressure in the surgical cavity and vent smoke and smoke plumes and gases at a predetermined rate to achieve optical clarity. The passive openings, e.g. multiple openings or flow restricted openings, can be configured, e.g., shaped and structured to provide a desired venting rate.

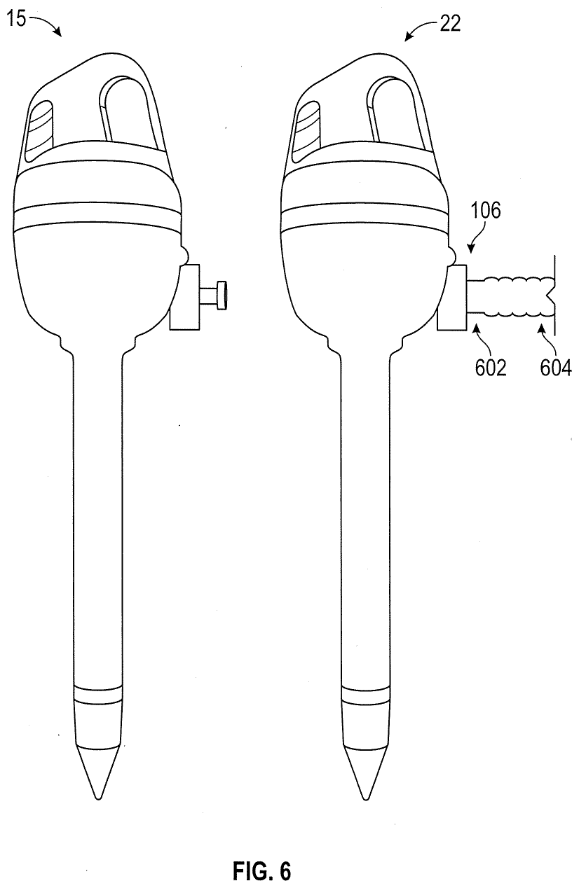

FIG. 6 illustrates a leak device 600 that includes a suction unit at the venting end 604. The suction unit can be coupled to a gases port 106 of a venting cannula 22 by the cannula connecting end 602 of the leak device 600 described above. A separate insufflation cannula 15 can be used for delivering insufflation fluid into the surgical cavity. The suction unit can include a filtering system, for example, described above. The suction unit can actively suck the gases from the surgical cavity via the venting cannula 22 into the filtering system. The filtered gases can diffuse into the atmosphere.

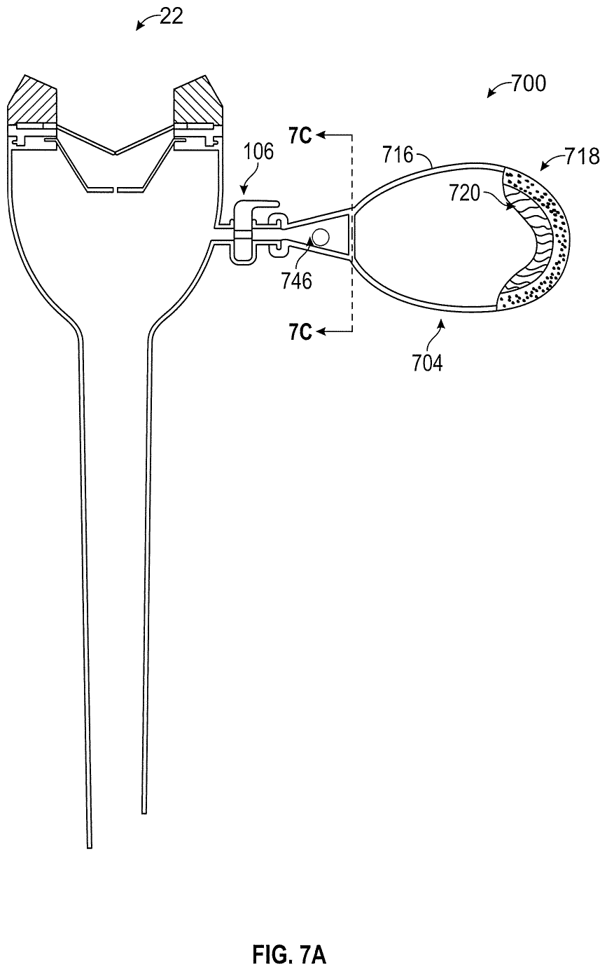

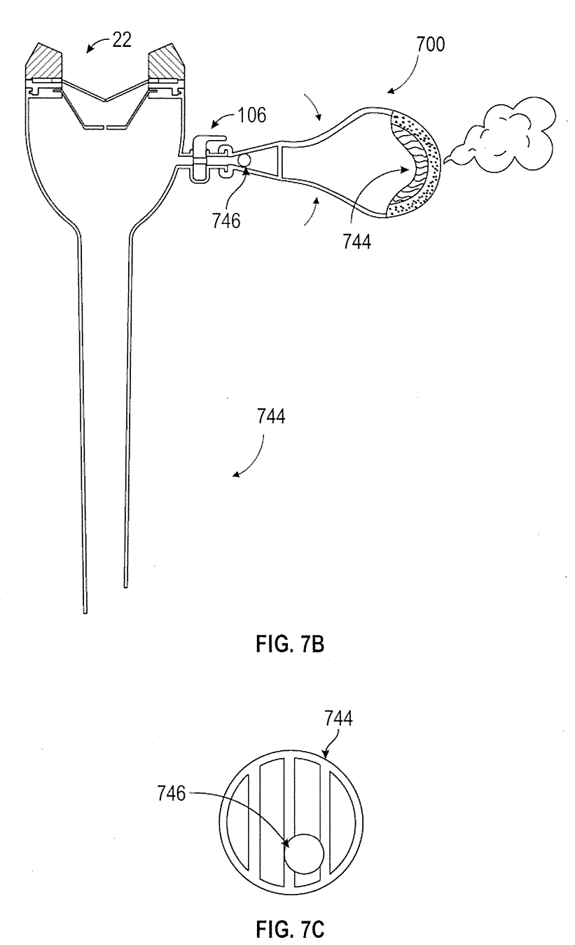

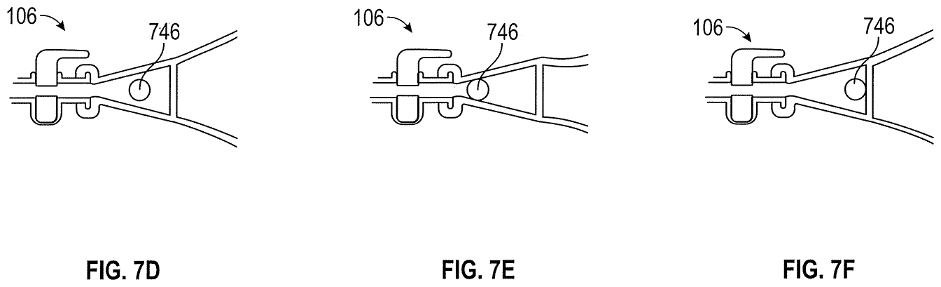

FIGS. 7A-7F illustrate a leak device 700 having a manual pump 704 at the venting end. The manual pump 704 can be configured to replace the suction unit of the leak device 600. As shown in FIG. 7A, the manual pump 704 can have the form of a venting sac lined by a gases impermeable lining 716 and a gases permeable membrane 718. The manual pump 704 can also be of other forms. The filter 720 (for example, ULPA and/or carbon filters) can be placed adjacent the gases permeable membrane 718. The device 700 can have any of the features of the device 300, 400, 500, 600 except as described herein.

As shown in FIG. 7B, the manual pump 704 can be squeezed by a user to diffuse the gases and/or smoke through the filter 720, and to expel the filtered gases and/or smoke through the gases permeable membrane 718. As shown in FIG. 7C, an opening of the manual pump 704 can include a plurality of slots 744, which can stop a ball 746 from leaving a valve location.

As shown in FIG. 7D, in normal operation when the manual pump 704 is not squeezed, pressure from the surgical cavity can push the ball 746 away from the gases port 106 to allow the manual pump 704 to be filled with the gases and/or smoke from the surgical cavity. As shown in FIG. 7E, when the manual pump 704 is squeezed, the ball 746 can be pushed proximally toward the gases port 106 of the cannula 22 to block more gases from exiting the cannula 22 before the current gases in the manual pump 704 is vented. As shown in FIG. 7F, when the pressure on the manual pump 704 is released, the ball 746 moves distally again and no longer blocks the entry of gases and/or smoke from the surgical cavity via the cannula 22 to the manual pump 704. A negative pressure is created inside the manual pump 704 so as to speed up evacuation of the gases and/or smoke from the surgical cavity into the manual pump 704.

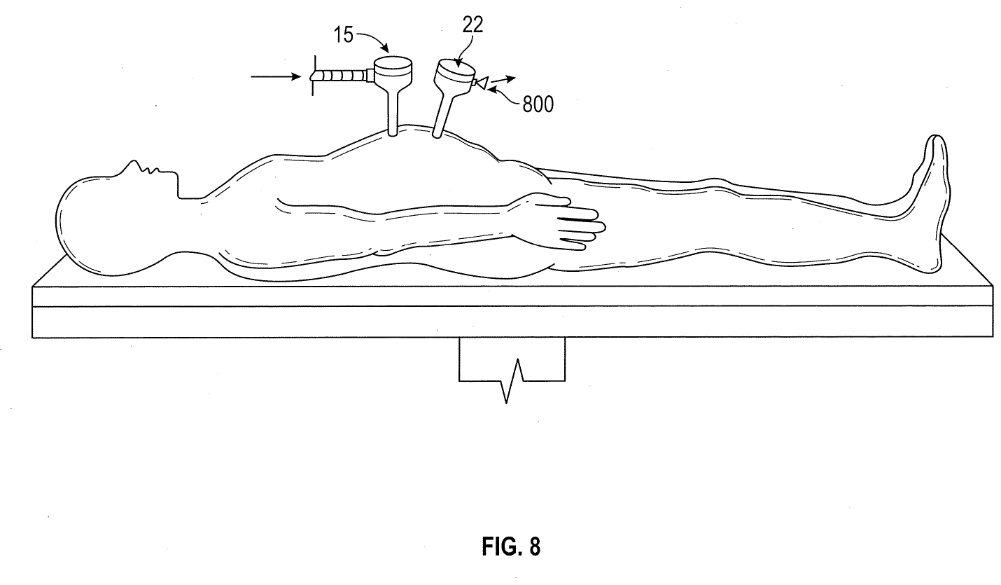

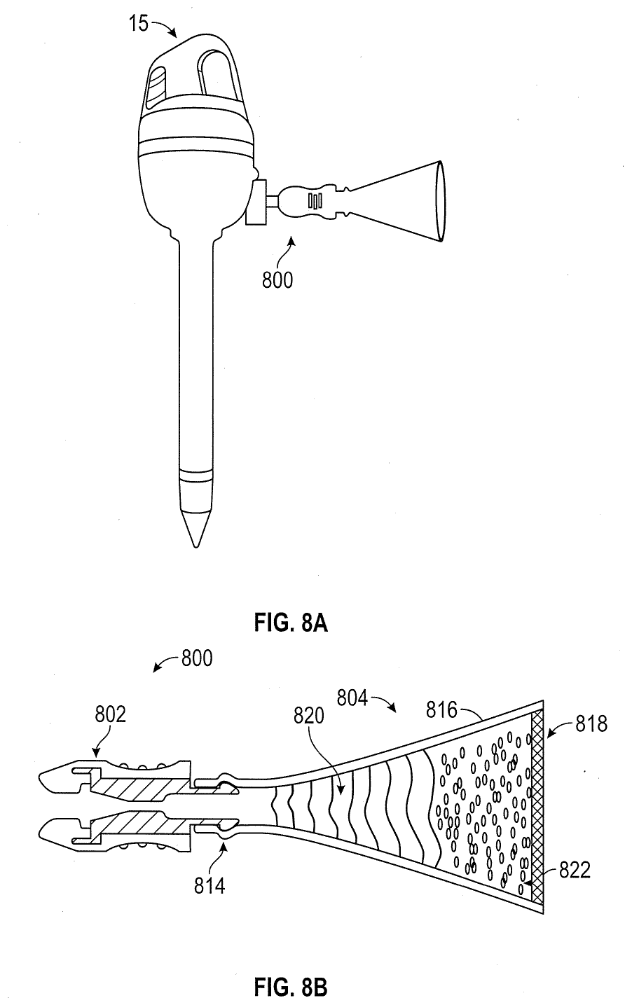

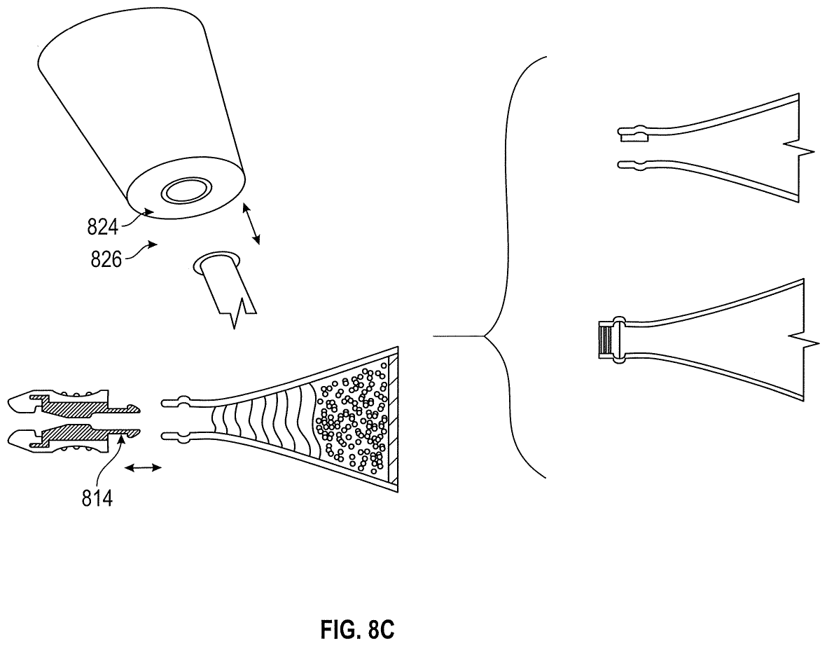

FIGS. 8 and 8A-8C illustrate a filter device 800 configured to be coupled to a venting cannula 22 by a cannula connecting end 802 described above. The filter device can be cone-shaped in some embodiments. As shown in FIG. 8, the venting cannula 22 can be a different cannula than the insufflation cannula 15 configured to deliver insufflation fluid into and/or allow a medical instrument to be inserted into the surgical cavity within the patient's body. The device 800 can have any of the features of the device 300, 400, 500, 600, 700.

As shown in FIG. 8B, the vent 804 of the device 800 can be coupled to the cannula connecting end 802 without a tube in between. The device 800 can include a flow restriction in the connector end 802, for example, a venting flow control device of FIG. 3A. The venting flow control device can have a flow restriction passage that is shaped and dimensioned in order to control the venting flow rate as described above. The vent 804 can have the cross-sectional diameter being greater at a distal end, where the gases permeable membrane 818 is located, than at a proximal end that is coupled to the barb 814 or any other suitable fixation methods, based on the disclosure herein, of the cannula connecting end 802. As an example, the filter device 800 can have a conical shape. The vent 804 can include the particulate filter 820 (for example, the ULPA filter) and optionally the odor filter 822 (for example, the carbon filter). The filter(s) can be overmoulded via Luer connection (see FIG. 8C) of the vent 800. The side wall of the vent 804 can be made of the gases impermeable lining 816.

As shown in FIG. 8C, the filter(s) can be removably coupled to the vent 804 via one or more of the Luer connection 824, 826, and/or with any other suitable ways of attaching the removable filter(s).

Examples of a Leak Device Attached to a Cannula Shaft

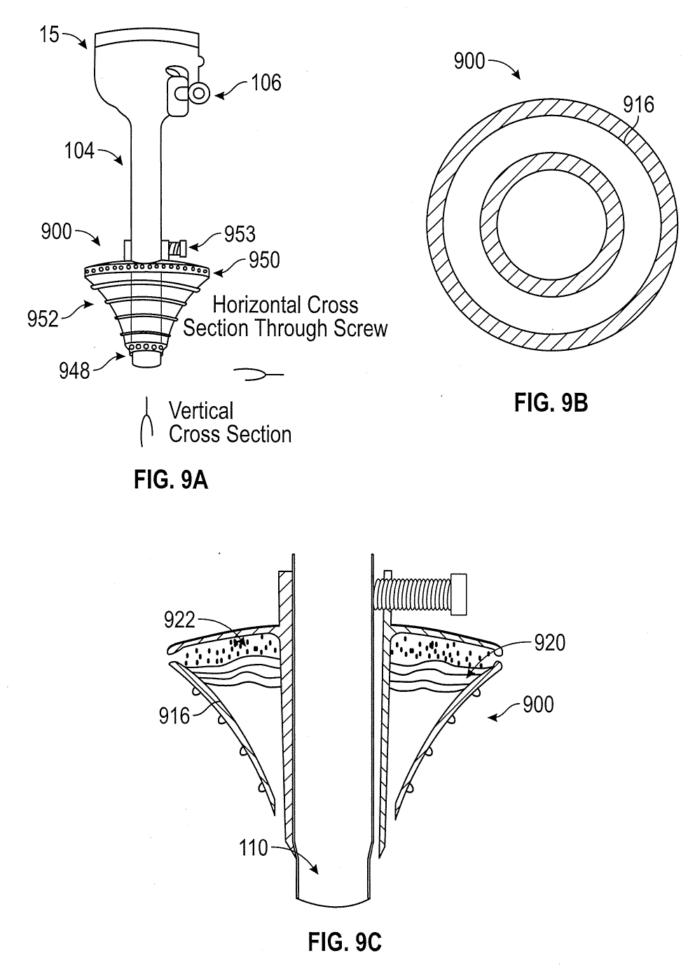

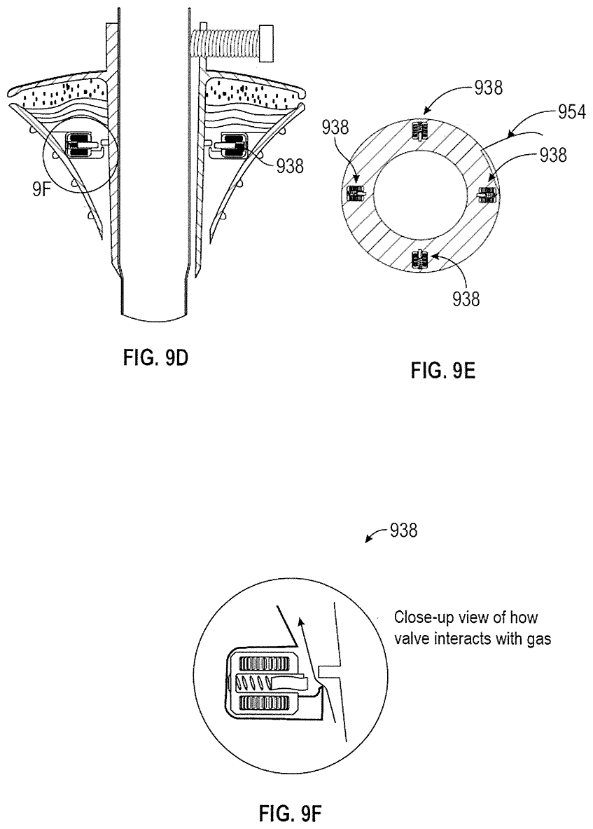

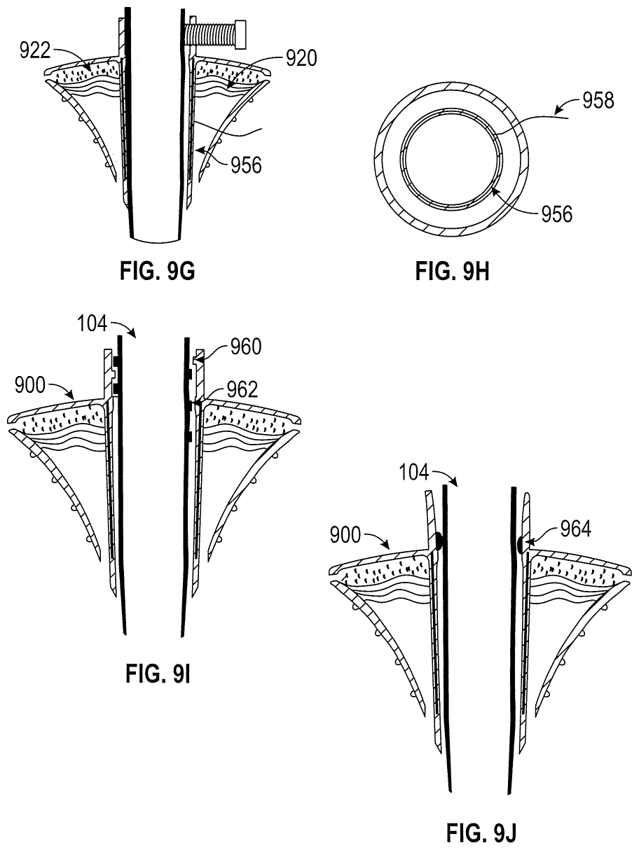

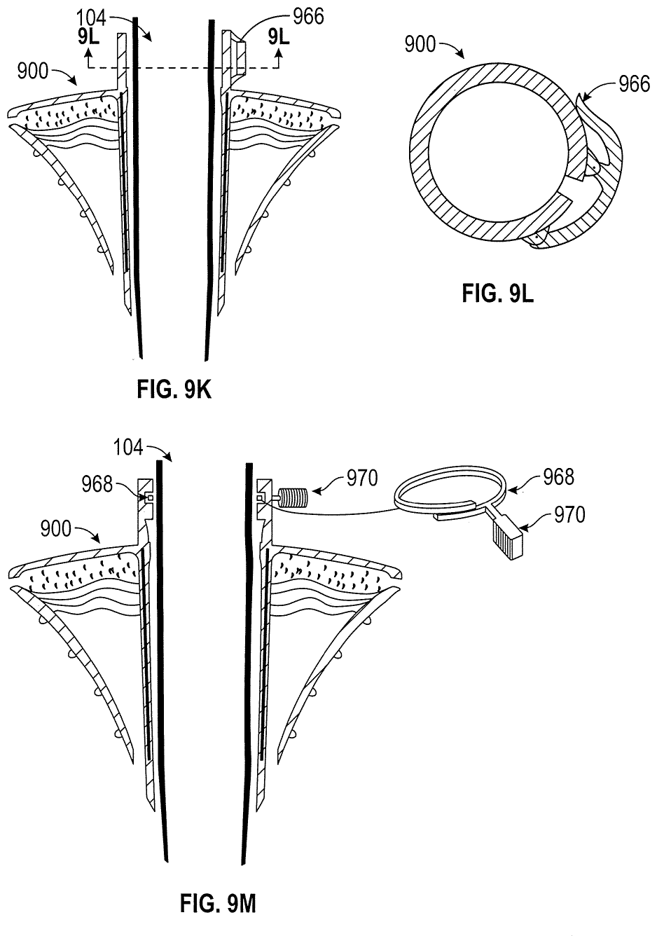

FIGS. 9A-9H illustrate examples of a sleeve leak device 900 configured to be coupled to an insufflation cannula 15. The sleeve leak device 900 can act as a leak device and/or to retain, locate, and/or control an insertion depth of the cannula during a surgical procedure. As shown in FIG. 9B-9C, the insufflation cannula 15 can include a lumen 110 configured to deliver insufflation fluid (for example, received via the gases port 106) into and/or allow a medical instrument to be inserted into the surgical cavity. As the leak device 900 does not obstruct the gases delivery path, the cannula 15 can be used for both insufflation fluid delivery and venting.

The sleeve leak device 900 can have an outer shape of generally a funnel. The leak device 900 can include a plurality of ridges 952 on an outer surface of the device 900. The ridges 952 can assist in retaining and repositioning the cannula 15 and/or sleeve leak device 900 within the surgical cavity. The ridges 952 can also help in retaining the cannula 15 in place. As described above, the sleeve leak device 900 can also act as a passage through the skin to allow manipulation of the cannula insertion depth.

The sleeve leak device 900 can include a lumen configured to slidably receive the elongate shaft 104 so that the sleeve leak device 900 circumferentially surrounds a portion of the elongate shaft 104, for example, near a distal end of the cannula 15. The sleeve leak device 900 can be securely attached to and/or repositioned on the elongate shaft 104 by a set screw 953, which can provide a radial pressure against the outer surface of the elongate shaft 104 when the screw 954 is tightened onto the elongate shaft 104. Other securement features can also be used to secure the sleeve leak device 900 to the shaft 104, such as shown in FIGS. 9I-9M.

As shown in FIG. 9I, threads 960 on an inner surface of the device 900 can mate with corresponding threads 962 along at least a portion of the shaft 104 of the cannula. The inner surface of the device 900 can have female threads 960 that mate with corresponding male threads 962 on the shaft 104. Alternatively, the inner surface of the device 900 can have male threads that mate with corresponding female threads on the shaft 104.

As shown in FIG. 9J, a protrusion 964 on an inner surface of the device 900 can be used. The protrusion 964 can extend partially (for example, in the form of a segment or multiple segments, or a dome-like bump or multiple bumps) or entirely (for example, in the form of a ring), along the inner surface of the device 900. The protrusion 964 can provide grip onto the shaft 104 of the cannula. The device 900 may be removed by applying a sufficient downward force to overcome the friction force between the protrusion 964 and the shaft 104 of the cannula, or by a generally radially outward force onto the wall of the device 900 to move the protrusion 964 radially outward and away from the shaft 104.

As shown in FIGS. 9K and 9L, a hinge 966 can be used to reversibly secure the device 900 onto the shaft 104 of the cannula. When the hinge 966 is closed, the device 900 can be frictionally locked onto the shaft 104 of the cannula. When the hinge 966 is opened, the device 900 can be removed from the shaft 104 of the cannula.

As shown in FIG. 9M, a spring lock 968 can be used. The spring lock 968 can form a ring or substantially a ring such that without an external force (that is, when the spring lock 968 is relaxed), the spring lock 968 can have an inner diameter configured to lock the device 900 onto the shaft 104 of the cannula. When an external force is applied to extend the spring lock, that is, to increase the inner diameter of the spring lock 968, the device 900 can be removed from the cannula. The spring lock 968 can be made of metal. As shown in FIG. 9M, the external force can be applied via a lever 970. The lever 970 can be applied in a counter-clockwise direction in the illustrated example to extend the spring lock 968. The spring lock 968 can be extended using any other suitable methods and/or tools based on the disclosure herein.

The sleeve leak device 900 can include one or more distal vents 948 and one or more proximal vents 950 to provide venting of the gases and/or surgical smoke, with a remainder of the sleeve leak device wall 916 being gases impermeable (for example, being made of plastic, for example, acrylonitrile butadiene styrene (ABS), polycarbonate (PC), and the like. In some examples, the wall 916 can have a thickness of about 0.1 mm to about 3 mm, or about 1 mm. The thickness of the wall 916 can vary. When in use, the leak device 900 can be partially inserted into the surgical cavity, with the distal vents 948 within the surgical cavity and the proximal vents 950 outside the patient's body. Gases and/or smoke in the surgical cavity can enter a space within the leak device 900 via the distal vents 948. The gases and/or smoke can diffuse upward through the particulate filter 920 and optionally the odor filter 922 before exiting through the proximal vents 950. The filter(s) can be located near a proximal end of the sleeve leak device 900, for example, adjacent the proximal vents 950 so that the gases and/or smoke are filtered before being released into the atmosphere. The leak device 900 can also optionally include one or more mechanical or passive venting valves (for example, an umbrella pressure relief valve described above) near a proximal end of the leak device 900 at a location after the gases and/or smoke have been filtered. The mechanical valves can be located at the small orifices, for example, at the proximal vents 950. Closing of the valve can restrict the orifice as described above. The valve(s) 938 can be selectively opened or closed (for example, to control the venting or leak rate).

As shown in FIGS. 9D-9F, the sleeve leak device 900 can also optionally include at least one (for example, two, three, four, or more) electrical valves 938 (for example, solenoid gate valves controlled by a PCB as described above) in a gases pathway in the leak device 900. The valve 938 can be located distal of the filter(s). The valves 938 can be in electrical communication with a controller (for example, the PCB in the humidifier, insufflator, or an external controller via wireless communication) via a wire 954 to control the venting or leak rate. The wire 954 can be moulded into the wall of the sleeve leak device 900. The wire 954 can also be used to connect a flow rate and/or smoke sensor. The valves 938 may be mechanical valves. The valves 938 may be included into other embodiments disclosed herein. The valves 938 can allow for controlling venting flow rate. The valves may be an alternative to the venting flow control device of FIG. 3A. The flow rate and/or smoke sensor can be moulded into the wall of the leak device 900.

As shown in FIGS. 9G and 9H, the sleeve leak device 900 can include a heating element (for example, a flexible PCB heater) 956. The other examples of venting attachment or leak device can also include a heating element. The heating element 956 can be located within the wall (for example, an inner wall) of the sleeve leak device 900. The heating element 956, when embedded in the wall of the device 900, does not come into contact with the gases path in the leak device 900 and/or the gases delivery lumen 110 to avoid contamination of the gases by the heating element 956. The heating element 956 can be configured to heat the filter(s) 920, 922 in order to reduce and/or prevent condensation and clogging in the filter(s), which can extend the life of the filter and aid in maintaining efficiency of the filter. The heating element 956 can be powered by the PCB via a wire connection 958.

The heaters can be incorporated into the filter elements. Further heating elements can be included in a shaft or a gases pathway. The heating element can be configured to heat the vented gases in order to reduce and/or prevent condensation in the passage or filter. The heating elements may be a heater wire, for example, a spiral wound heater wire, a PCB heater, conductive ink, conductive polymers, or the like.

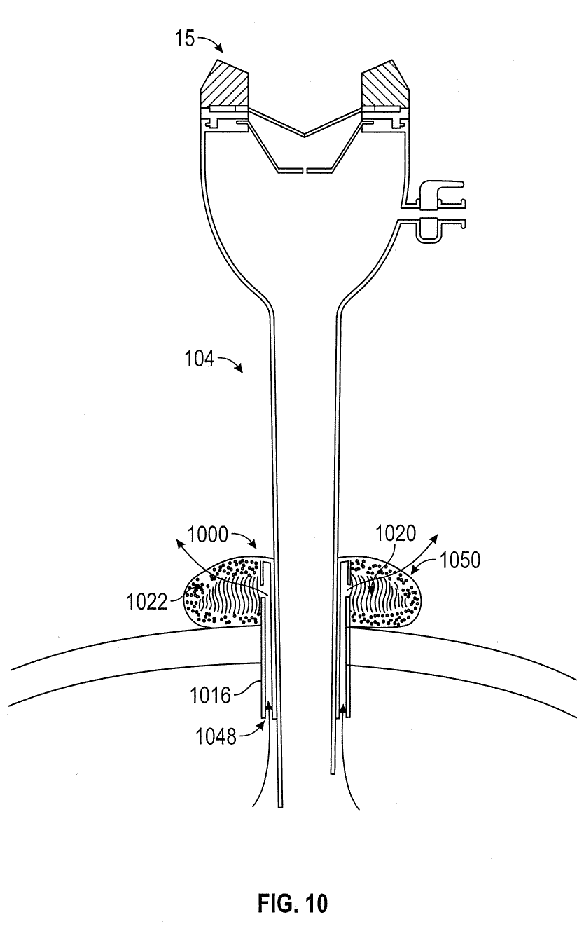

FIG. 10 illustrates a venting seal leak device 1000, which can be used instead of the sleeve leak device 900 to be coupled to the elongate shaft 104 of an insufflation cannula 15. The venting seal leak device 1000 can have any of the features of the sleeve leak device 900. The venting seal leak device 1000 can form a seal around the elongate shaft 104 at an opening of the surgical cavity. When in use, the leak device 1000 can be partially inserted into the surgical cavity, with the proximal vents 1050 outside the patient's body. Gases and/or smoke in the surgical cavity can enter a space within the leak device 1000 via a distal opening 1048. The gases and/or smoke can diffuse upward through the particulate filter 1020 and optionally the odor filter 1022 before exiting through the proximal vents 1050.

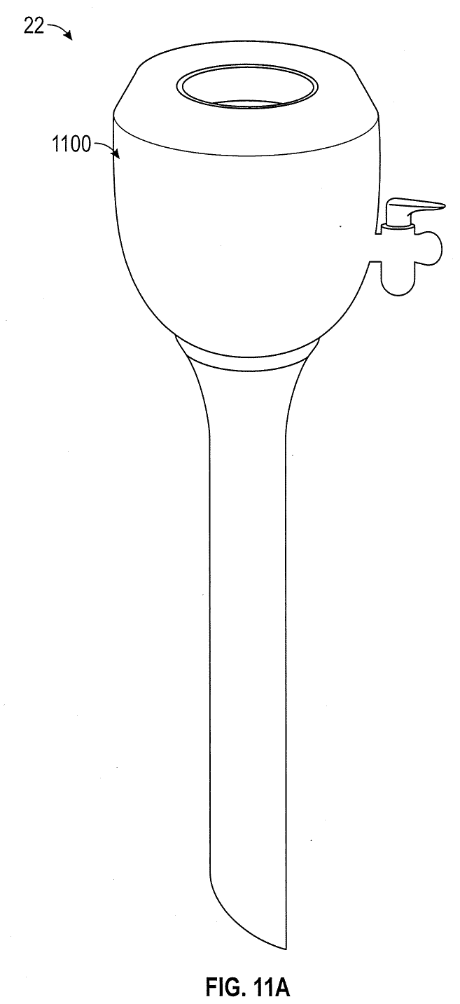

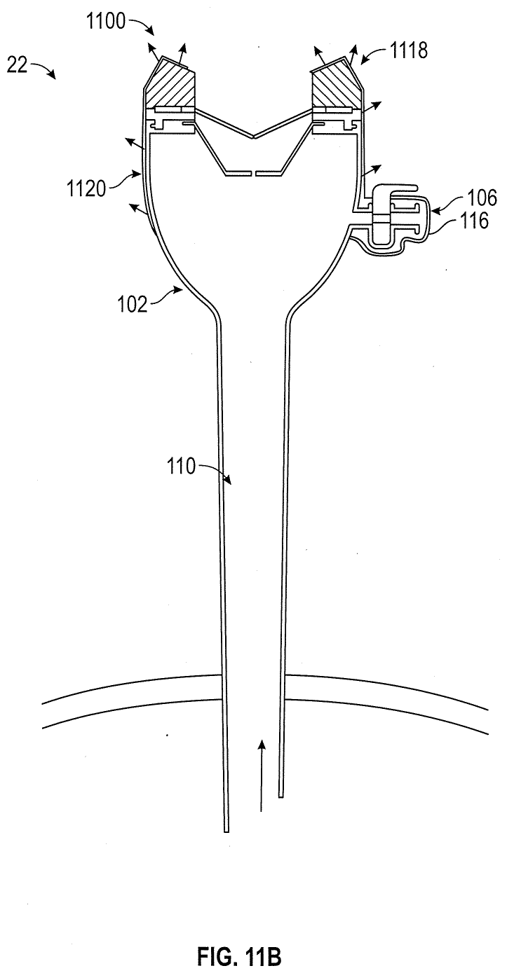

FIGS. 11A and 11B illustrate a sleeve cap leak device 1100, which can be used instead of the sleeve leak device 900 or the venting seal leak device 1000. The sleeve cap leak device 1100 can be applied to the upper housing 102 of a venting cannula 22 rather than a gases delivery cannula. The leak device 1100 can enclose the upper housing 102, including the gases port 106 with the gases impermeable lining 1116 and cover an outer surface of the upper housing 102 with a gases permeable membrane 1118. Gases and/or smoke entering the cannula lumen 110 from the surgical cavity is forced to diffuse through the filters (for example, ULPA and/or carbon filters) 1120 and the filtered gases can vent through the gases permeable membrane 1118.

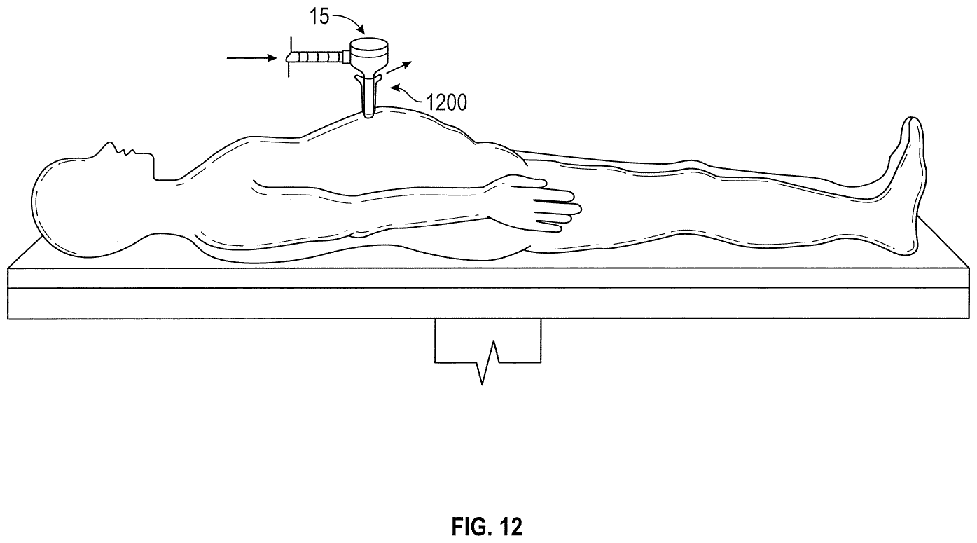

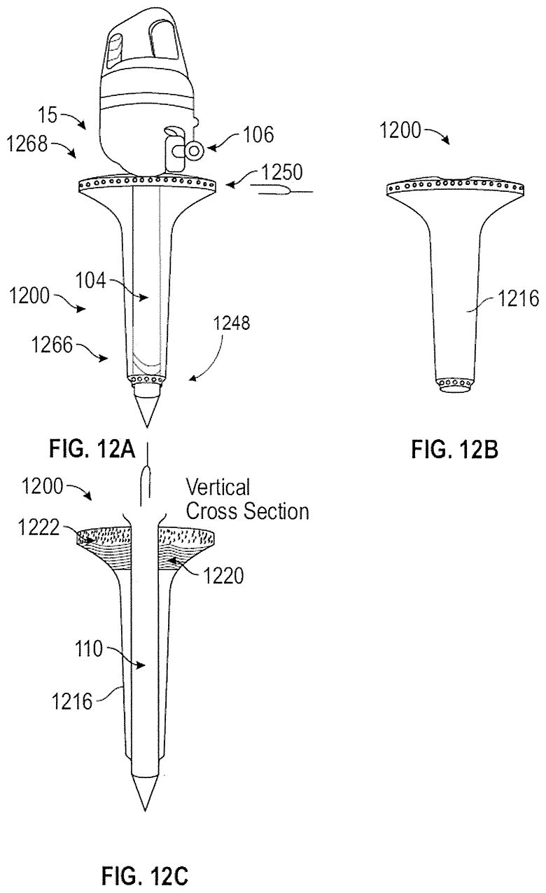

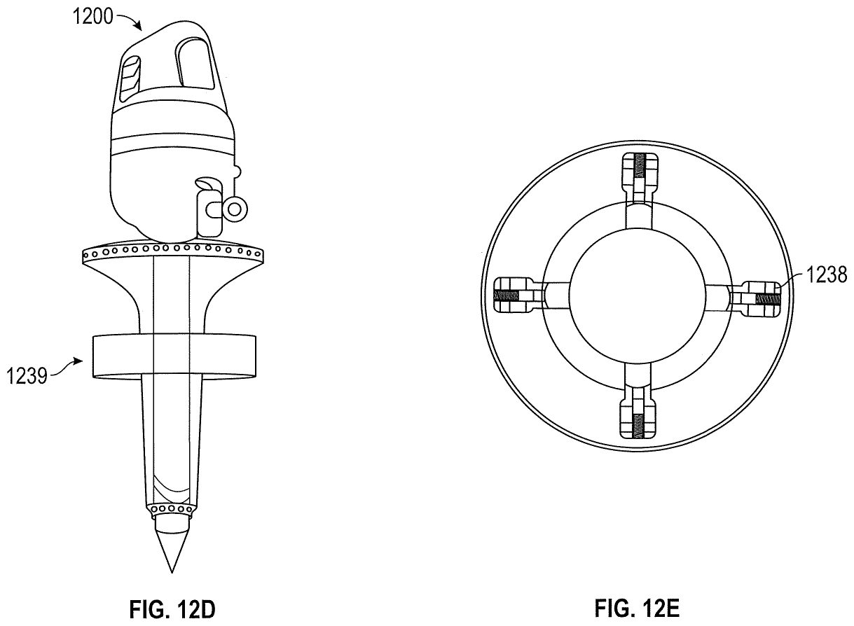

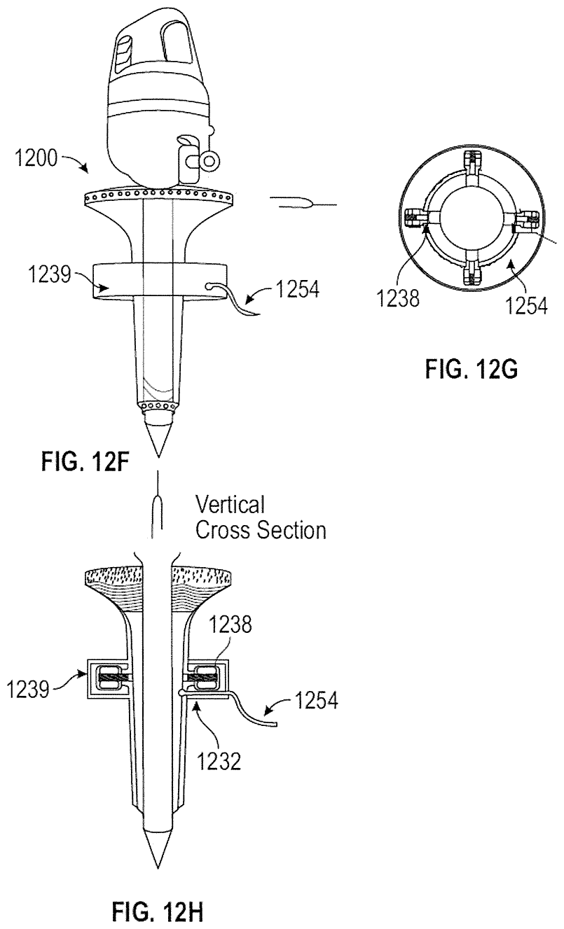

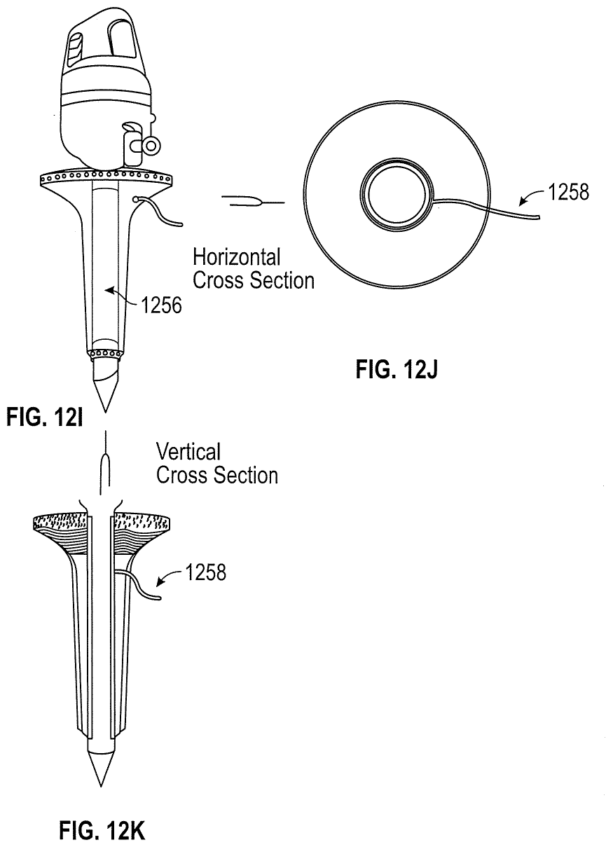

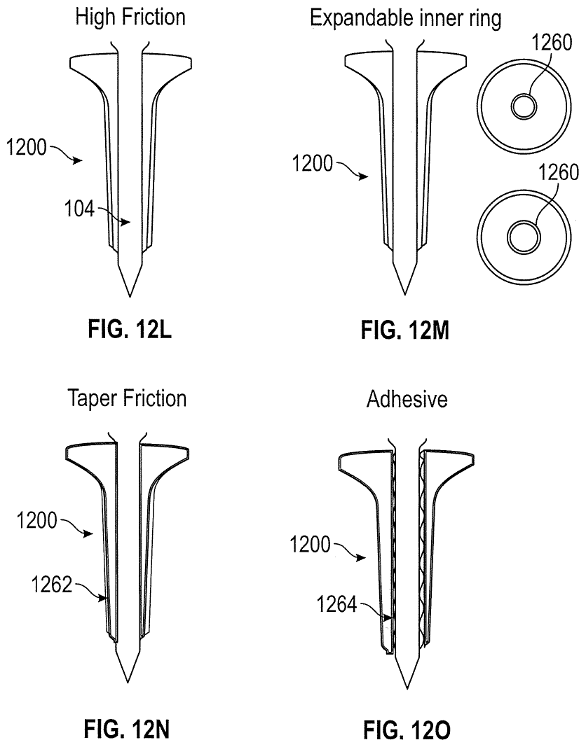

FIGS. 12 and 12A-12O illustrate a shaft sleeve leak device 1200 configured to be coupled to an insufflation cannula 15 (for example, having an inner diameter of about 3 mm to about 15 mm, or any other value) or another cannula, such as a venting cannula. The insufflation cannula 15 can include a lumen 110 configured to deliver insufflation fluid (for example, received via the gases port 106) into and/or allow a medical instrument to be inserted into the surgical cavity. As the leak device 1200 does not obstruct the gases delivery path, the cannula 15 can be used for both insufflation fluid delivery and venting. The shaft sleeve leak device 1200 can have any of the features of the sleeve leak device 900.

The shaft sleeve leak device 1200 can have a slim sleeve portion 1266 and an enlarged proximal portion 1268. The shaft sleeve leak device 1200 can include a lumen configured to slidably receive the elongate shaft 104 so that the shaft sleeve leak device 1200 circumferentially surrounds the elongate shaft 104 (for example, substantially along an entire length of the shaft 104). The enlarged proximal portion 1268 can be near or adjacent the upper housing of the cannula 15. A distal end of the slim sleeve portion 1266 can be near a distal end or outlet of the cannula 15.

The shaft sleeve leak device 1200 can include one or more distal vents 1248 and one or more proximal vents 1250 to provide venting of the gases and/or surgical smoke, with a remainder of the sleeve leak device wall being gases impermeable 1216 (for example, being made of plastic, for example, ABS and the like). When in use, the leak device 1200 can be partially inserted into the surgical cavity, with the distal vents 1248 within the surgical cavity and the proximal vents 1250 outside the patient's body.

Gases and/or smoke in the surgical cavity can enter a space within the leak device 1200 via the distal vents 1248. The gases and/or smoke can diffuse from the inside of the particulate filter 1220 to the outside of the particulate filter 1220, and optionally also through the odor filter 1222 before the filtered gases exit through the proximal vents 1250. The filter(s) can be located near a proximal end of the shaft sleeve leak device 1200, for example, adjacent the proximal vents 1250 and/or occupying substantially the proximal portion 1268, so that the gases and/or smoke are filtered before being vented. The proximal vents 1250 can include a plurality of small orifices, which can also optionally be used in combination with valves.

As shown in FIGS. 12D and 12E, the leak device 1200 can optionally include one or more mechanical or passive venting valves in a gases pathway in the leak device 1200. The mechanical valves 1238 can be located in a valve collar 1239 slidably disposed on the slim sleeve portion 1266. The valve 1238 can be located distal of the filter(s) but high enough so as to not interfere with the wound opening to the surgical cavity. As shown in FIGS. 12F-12H, the valve collar 1239 can also optionally house at least one (for example, two, three, four, or more) electrical valves 1238. The valves 1238 can be in electrical communication with a control system (for example, a PCB) via a wire 1254 so as to actuate the electrical valves 1238. Examples of mechanical and/or electrical valves 1238 used can include an umbrella pressure relief valve, a solenoid gate valve, a standard pressure relief valve, and/or a diaphragm pressure relief valve as described above. Each valve 1238 can include a small restricting gases path with a remainder of the shaft sleeve leak device 1200 in the same cross-section closed up to direct the gases to be vented through the paths in the valves 1238. The valve(s) 1238 can be selectively opened or closed (for example, to control the venting or leak rate).

As shown in FIGS. 12G and 12H, the wire 1254 can sit loosely in the valve collar 1254 and does not interfere with the valves 1238 and/or venting of the gases. The wire 1254 can be connected to a control system of the insufflator or humidifier unit. The wire 1254 can also split to connect to a flow rate and/or smoke sensor 1232, which can be moulded into the wall of the leak device 1200.

As shown in FIGS. 12I-12K, the shaft sleeve leak device 1200 can include a heating element (for example, a flexible PCB heater) 1256. The heating element 1256 can be located within the wall (for example, an inner wall) of the shaft sleeve leak device 1200. Locating the heating element 1200 in the inner wall can reduce harm to the patient when the cannula 15 and the leak device 1200 come into contact with the patient's skin. The heating element 1256 does not come into contact with the gases path in the leak device 1200 and/or the gases delivery lumen 110 so avoid contamination of the gases by the heating element 1256. The heating element 1256 can be configured to heat the gases passing through the leak device 1200 before the gases reach the filter(s) 1220, 1222. The heated gases can reduce and/or prevent condensation and clogging in the filter(s), which can extend the life of the filter and aid in maintaining efficiency of the filter. The heating element 1256 can be powered by the PCB via a wire connection 1258.

FIGS. 12L-12O illustrate methods of securing the shaft sleeve leak device 1200 to the elongate shaft 104 of a cannula. As shown in FIG. 12L, an inner diameter of the leak device 1200 can be designed to have a tight tolerance on the outer wall of the elongate shaft 104 to be secured by friction. As shown in FIG. 12M, the leak device 1200 can include an expandable inner ring 1260. The ring 1260 can be made of a flexible material, for example, nitrile or other materials. The inner diameter of the ring 1260 can be initially smaller than the outer diameter of the elongate shaft 104 of the cannula. In some examples, the inner diameter of the ring 1260 can be initially about 60% to about 85%, or about 75% smaller than the outer diameter of the elongate shaft 104. The ring 1260 can be expanded or stretched to be placed on the elongate shaft 104. The restoring forces of the elastic material of the inner ring 1260 can maintain the shaft sleeve leak device 1200 on the elongate shaft 104 of the cannula. As shown in FIG. 12N, the shaft sleeve leak device 1200 can have a taper on the inner wall 1262 so as to lock the leak device 1200 on the elongate shaft 104 of the cannula by friction. As shown in FIG. 12O, an adhesive 1264 (for example, glue, adhesive tape, and the like), straps, clamps, or others can be used to secure the leak device 1200 to the elongate shaft 104 of the cannula.

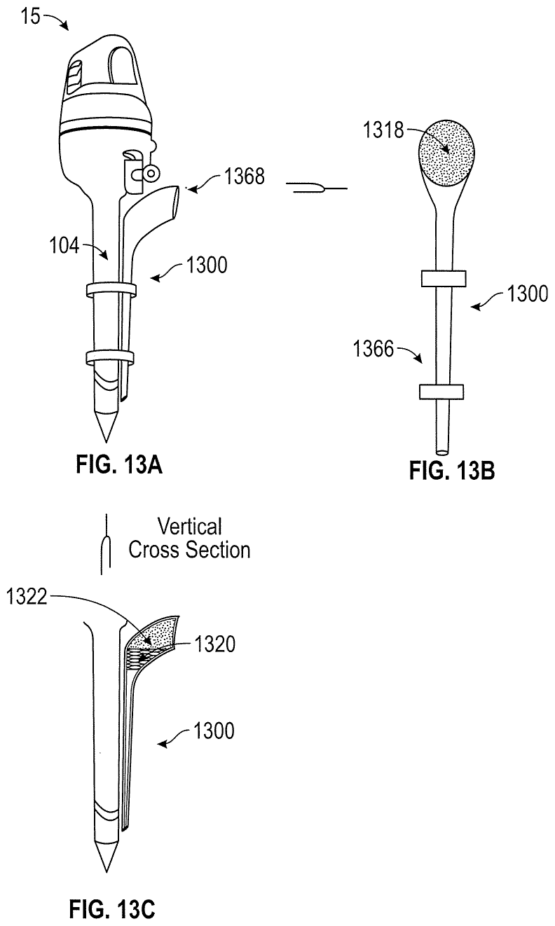

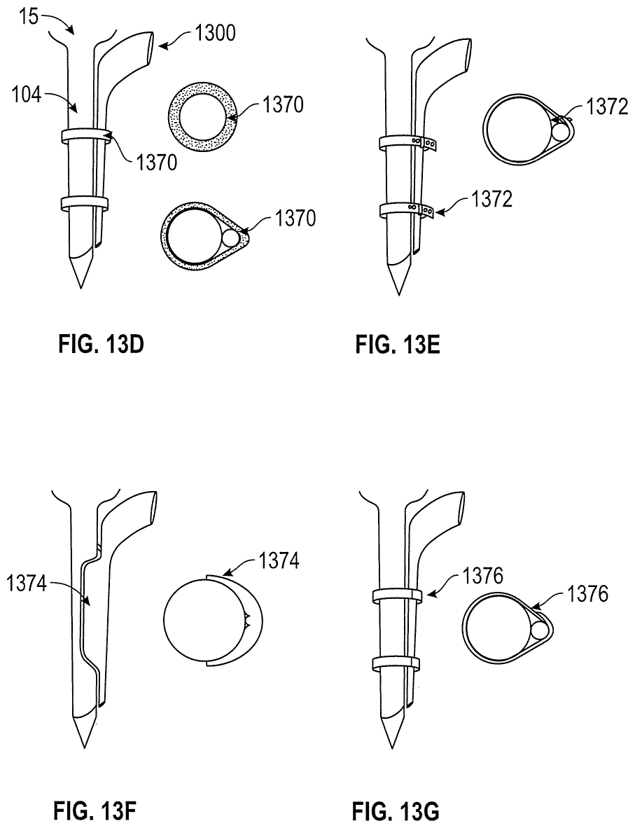

FIGS. 13A-13G illustrate a leak device 1300 that can be used to replace the shaft sleeve leak device 1200. The leak device 1300 can have a shaft 1366 and an enlarged venting end 1368 located at a proximal end of the shaft 1366. The leak device 1300 can have any of the features of the shaft sleeve leak device 1200 except that the shaft 1366 includes a single-lumen tube that does not completely surround the cannula shaft 104, whereas the slim sleeve portion 1266 of the shaft sleeve leak device 1200 includes a radial sleeve that completely or substantially completely surrounds the cannula shaft 104. The venting end 1368 can contain the filters 1320, 1322 (for example, the ULPA and/or carbon filters). As shown in FIGS. 13B and 13C, the filters 1320, 1322 can be located next to a plurality of small outlets 1318 to vent the filtered gases.

As shown in FIG. 13D, the leak device 1300 can be secured to the elongate shaft 104 of the cannula 15 by one or a plurality of (for example, two, three, or more) flexible and/or stretchable bands 1370. The band can be made from nitrile or the like material. The inner diameter of the band 1370 can be initially smaller than the outer diameter of the elongate shaft 104 of the cannula. In some examples, the inner diameter of the band 1370 can be initially, for example, about 60% to about 85%, or about 75% smaller than the outer diameter of the elongate shaft 104. The band 1370 can be expanded or stretched to be placed over the elongate shaft 104 and the shaft 1366 of the leak device 1300. The restoring forces of the elastic material of the band 1370 can maintain the leak device 1300 on the elongate shaft 104 of the cannula.

As shown in FIG. 13E, the leak device 1300 can be secured to the elongate shaft 104 of the cannula 15 by one or a plurality of (for example, two, three, or more) bands having belt and/or cable tie mechanisms 1372. Tightness of the band can be adjusted as desired.

As shown in FIG. 13F, the shaft 1366 of the device 1300 can include a plurality of wings 1374 that extend outwardly from a body of the device 1300. The wings 1374 can be flexible. The wings 1374 can extend outward from the shaft 1366 in the opposite direction as the enlarged venting end 1368. The wings 1374 can extend along at least a portion of, or substantially an entirety of the shaft 1366. The wings 1374 can wrap around the elongate shaft 104 of the cannula and can be secured by an adhesive (for example, glue, adhesive tape, or the like) to the elongate shaft 104. In some configurations, the wings 1374 may have a width such that the two wings can clip together around the shaft 104 of the cannula.

As shown in FIG. 13G, the leak device 1300 can be secured to the elongate shaft 104 of the cannula 15 by a plurality of (for example, two, three, or more) adhesive bands 1376.

In other configurations, the device 1300 can be coupled to the cannula shaft 104 using any suitable fastener.

Examples of a Leak Device Attached to a Cannula Upper Housing

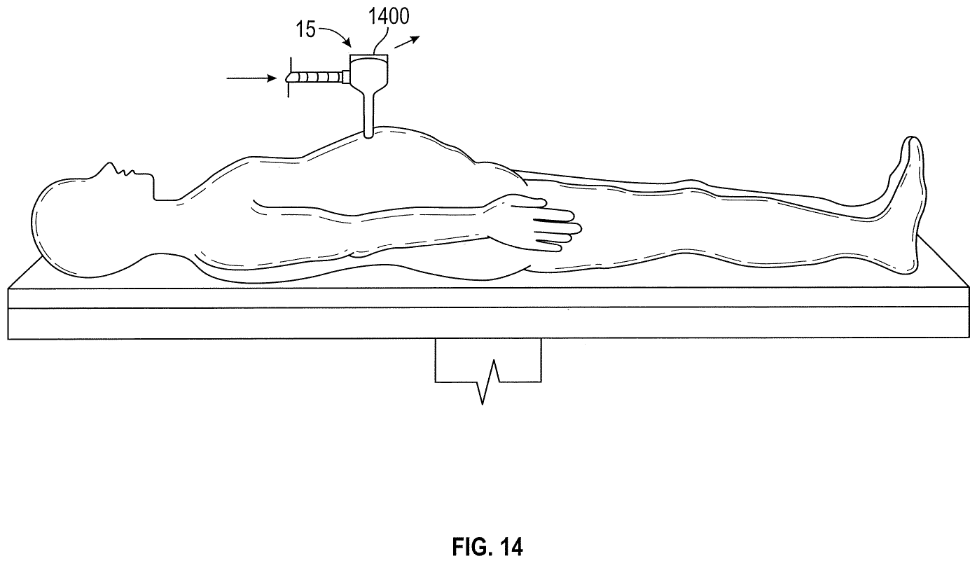

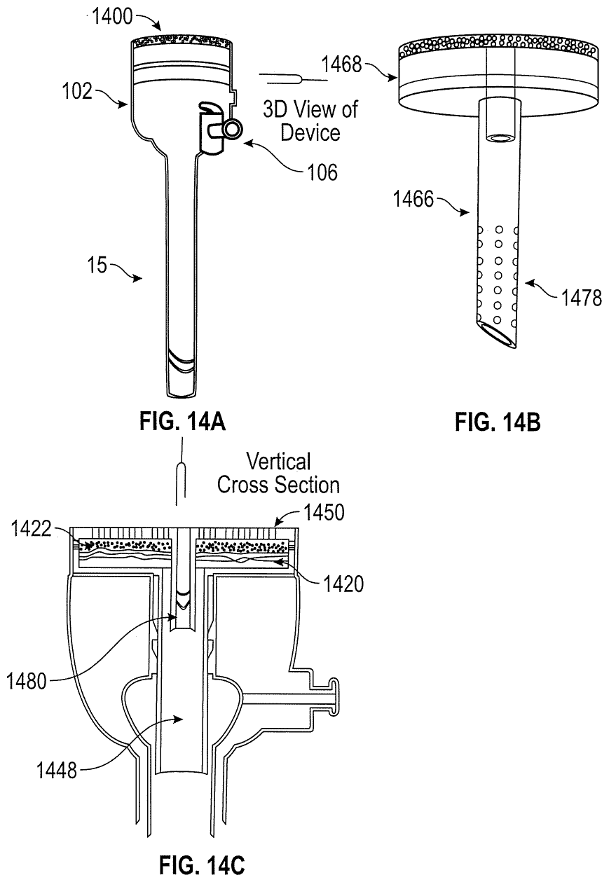

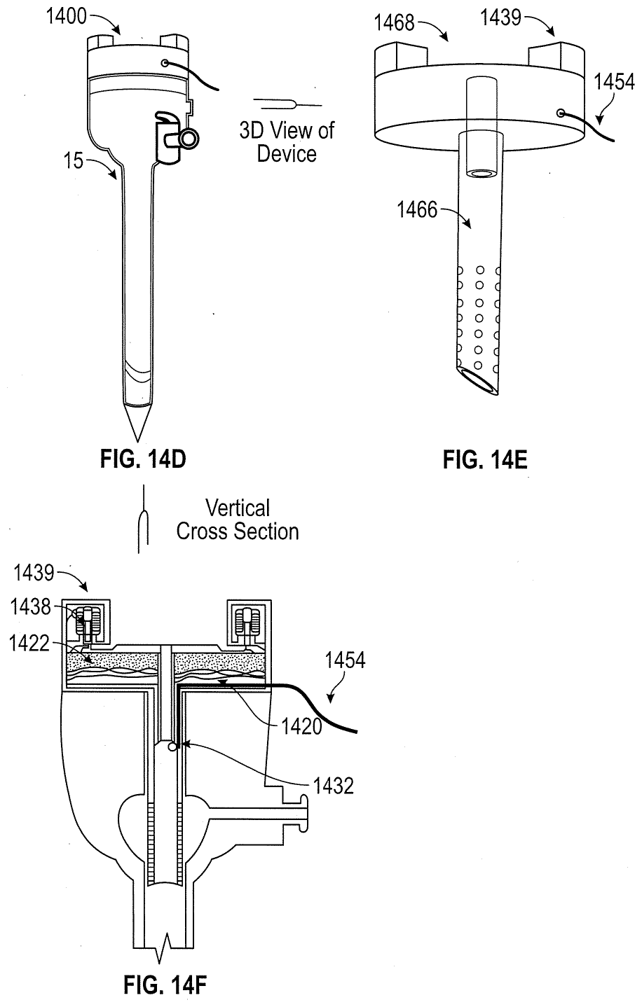

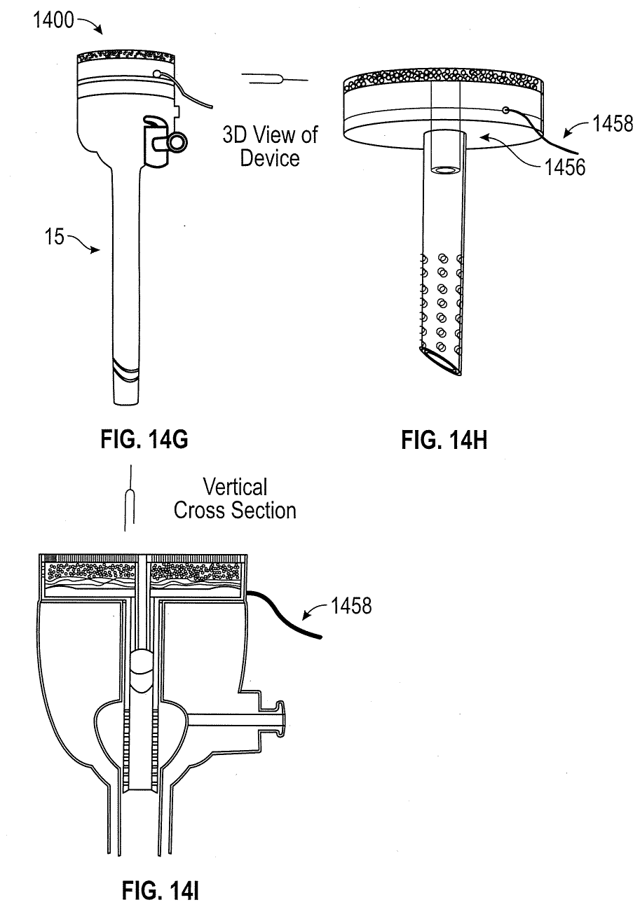

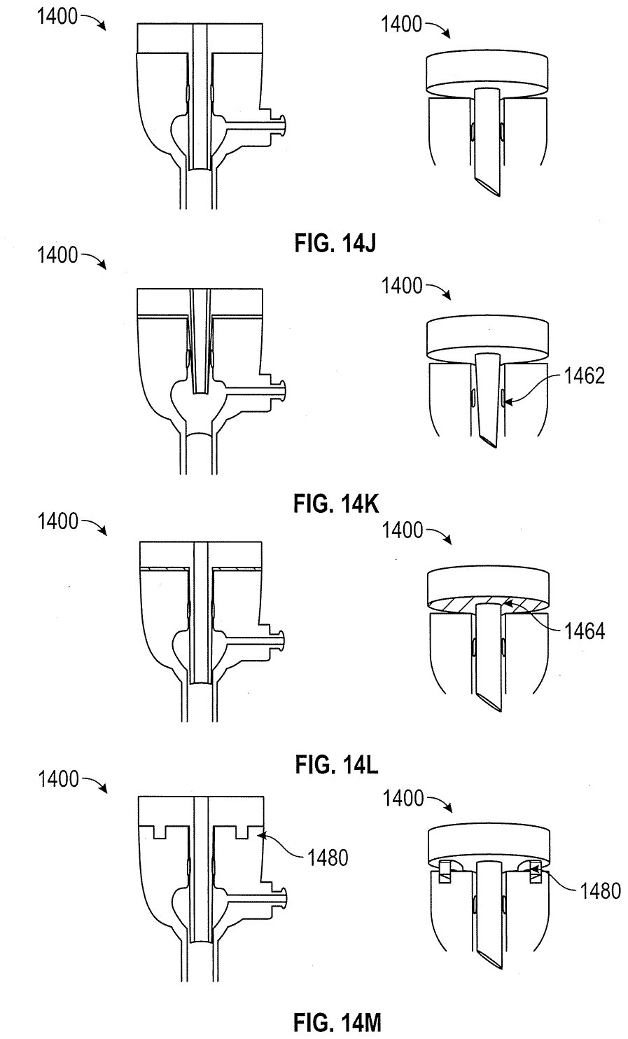

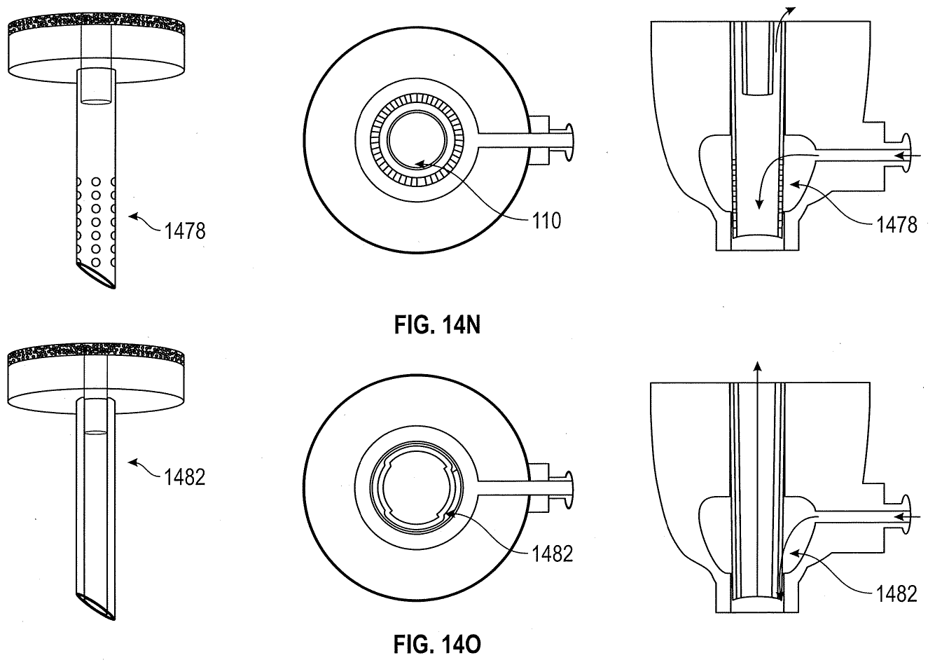

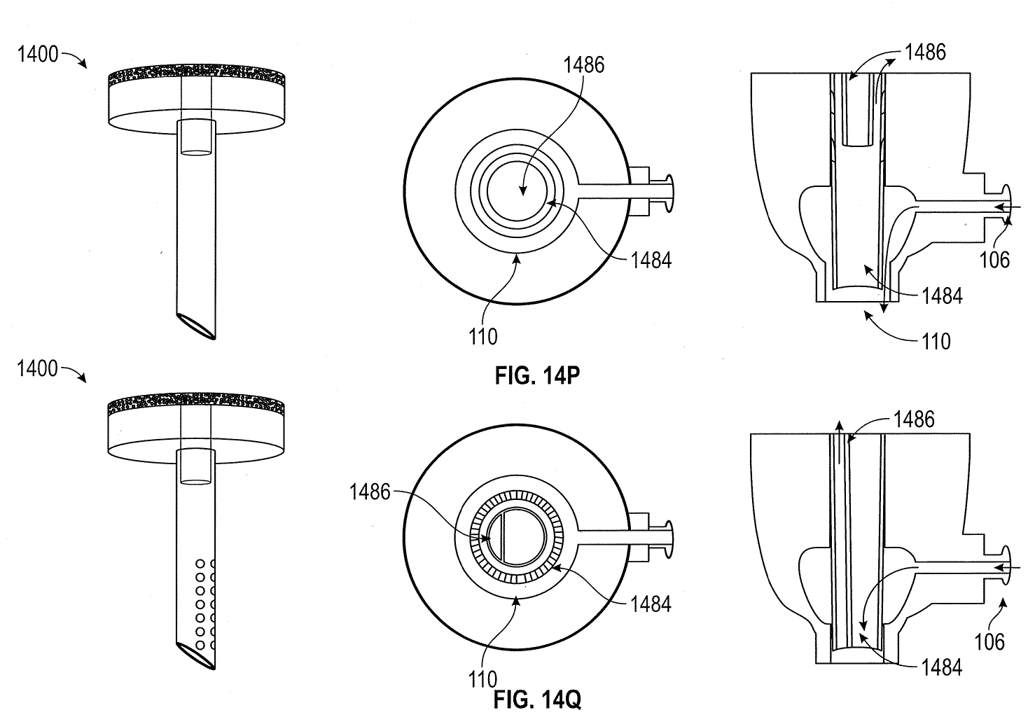

FIGS. 14 and 14A-14Q illustrate an insertable filter device 1400 configured to be coupled to a cannula, for example, an insufflation cannula 15. The insufflation cannula 15 can include a lumen 110 configured to deliver insufflation fluid (for example, received via the gases port 106) into and/or allow a medical instrument to be inserted into the surgical cavity. The insertable filter device 1400 can provide a pathway for gases to exit through the cannula 15 after being filtered and does not obstruct the lumen 110 for delivery of the insufflation fluid. In some configurations, the device is not coupled to the insufflation cannula 15.

As shown in FIGS. 14A-14C, the insertable filter device 1400 can have a sleeve portion 1466 configured to be inserted through a proximal end of the cannula 15 into the upper housing 102 of the cannula 15. The insertable filter device 1400 can have an enlarged proximal base 1468 configured to remain proximal to the upper housing 102 of the cannula 15. The proximal base 1468 can be disc-shaped and/or sized to substantially match the shape of the cannula upper housing 102. The proximal base 1468 can have different shapes and/or sizes. The sleeve portion 1466 can terminate at or near where the upper housing 102 transitions to the elongate shaft 104 of the cannula 15. The sleeve portion 1466 can include one or more holes 1478 configured for gases entry into the cannula lumen 110 from the gases port 106.

The sleeve portion 1466 can be made of a gases impermeable material (for example, being made of plastic, for example, ABS, PC, and the like). In some configurations, the material 1416 can have a thickness of about 0.4 mm to about 0.8 mm, or about 0.6 mm. The sleeve portion 1466 can have an open lumen 1448 and the enlarged proximal base 1468 can include one or more proximal vents 1450 to provide venting of the gases and/or surgical smoke. When in use, gases and/or smoke from within the surgical cavity can enter the open lumen 1448 of the sleeve portion 1466 and travel upward to the proximal vents 1450. The gases and/or smoke can diffuse through the particulate filter 1420 and optionally the odor filter 1422 (see FIG. 14F) before exiting through the proximal vents 1450. The filter(s) can be located near the proximal vents 1450 so that the gases and/or smoke are filtered before being vented. The proximal vents 1450 can include a plurality of small orifices, which can optionally be used in combination with valves (for example, the mechanical and/or electrical valves) for example, describe above.

The lumen 1448 of the sleeve portion 1466 can also guide a medical instrument (for example, a scope) when inserted into the cannula 15. The insertable filter device 1400 can also include an instrument seal (for example, a duckbill seal) 1480 to provide sealing of the gases and/or smoke inside the insertable filter device 1400. The seal 1480 can conform around the medical instrument inserted into the cannula 15 via the insertable filter device 1400 to prevent gases from escaping in spaces surrounding the medical instrument.

The insertable filter device 1400 can optionally include one or more mechanical or passive venting valves in a gases pathway in the insertable filter device 1400, for example, to replace or be used in combination with the small orifices at the proximal vents 1450. As shown in FIGS. 14D-14F, the valves can also optionally include electrical valves 1438 in a gases pathway in the device 1400. The electrical valves 1438 can be in electrical communication with a control system (for example, a PCB) via a wire 1454 so as to actuate the electrical valves 1438. Examples of valves 1438 used can include an umbrella pressure relief valve, a solenoid gate valve, a standard pressure relief valve, and/or a diaphragm pressure relief valve as described above. In some configurations, the insertable filter device 1400 can be configured to maintain a pressure in the surgical cavity under about 50 mmHg and more preferably under about 30 mmHg.

As shown in FIG. 14F, the valves 1438 can be located at a proximal end of the device 1400. The valves 1438 can be covered by casing 1439. Each valve 1438 can include a small restricting gases path with a remainder of the device 1400 in the same cross-section as the valves 1438 open to direct the gases through the paths in the valves 1438. The valve(s) 1438 can be selectively opened or closed (for example, to control the venting or leak rate).

As shown in FIG. 14F, the wire 1454 can extend between the filter 1420 and a wall of the proximal base 1468. The wire 1454 can be connected to a control system of the insufflator or the humidifier unit or another controller. The wire 1454 can also split to connect to a flow rate and/or smoke sensor 1432 or any other suitable sensor, which can be moulded into the wall of the sleeve portion 1466 of the leak device 1400.

As shown in FIGS. 14G-14I, the insertable filter device 1400 can include a heating element (for example, a flexible PCB heater) 1456. The heating element 1456 can be located on a bottom surface of the proximal base 1468 of the insertable filter device 1400. The heating element 1456 can be configured to heat the gases passing through the device 1400 before the gases reach the filter(s) 1420, 1422. The heated gases can reduce and/or prevent condensation and clogging in the filter(s), which can extend the life of the filter and aid in maintaining efficiency of the filter. The heating element 1456 can be powered by the PCB via a wire connection 1458. The heating element 1456 can be integrated with the electrical valves 1438 such that the wire 1454 and the wire 1458 can be the same wire. The heating element 1456 can also be a standalone feature such that the wire 1454 and the wire 1458 are different wires.

FIGS. 14J-14M illustrate methods for securing the insertable filter device 1400 to the upper housing 102 of a cannula. As shown in FIG. 14J, an inner diameter of the device 1400 can be designed to have a tight tolerance with the standard cannula seals to be secured by friction. As shown in FIG. 14K, the device 1400 can have a taper 1462 on the sleeve portion 1466 so as to interact with the cannula seals to wedge the insertable filter device 1400 in place. The device 1400 can be inserted by force into the cannula seals. As shown in FIG. 14L, an adhesive 1464 (for example, glue, adhesive tape, and the like) can be used to secure the bottom of the proximal base 1468 to a top surface of the cannula. The adhesive may be covered by a protective liner. A user can peel off the liner to reveal the adhesive and press the insertable filter device 1400 onto a cannula. The adhesive can also be applied to the device 1400 before pressing the device 1400 onto the cannula. As shown in FIG. 14M, the insertable filter device 1400 can include locking features 1480 designed to lock the device 1400 by pressing the locking features 1480 into corresponding features on the cannula.

As shown in FIG. 14N, the one or more holes 1478 on the sleeve portion 1466 can be configured to allow gases entry into the cannula lumen 110 from the gases port 106, which may be otherwise be blocked by a portion of the sleeve portion 1466. The insertable filter device 1400 can have double concentric lumens 1484, 1486 (see FIG. 14P) so that the gases being vented diffuse into the filters between the double lumen walls. If the insertable filter device 1400 is coupled with an insufflation cannula, the gases can vent during the pauses in pulsatile insufflation. If the insertable filter device 1400 is not coupled to an insufflation cannula, the gases can simply vent out. As shown in FIG. 14O, instead of the holes 1478, the sleeve portion 1466 can include indents 1482 to allow gases entry into the cannula lumen 110 from the gases port 106. The gases being vented can leave from the single lumen of the sleeve portion 1466. As shown in FIG. 14P, the insertable filter device 1400 can have double concentric lumens 1484, 1486 so that the gases being vented diffuse into the filters between the double lumen walls. The outer lumen 1484 of the sleeve portion 1466 can have a smaller diameter than the cannula lumen 110 to allow gases entry from the gases port 106 to the cannula lumen 110. The smaller sleeve portion 1466 can be sealed and/or held in a concentric arrangement relative to the cannula lumen 110 by the cannula seals. As shown in FIG. 14Q, the insertable filter device 1400 can have double offset lumens and the one or more holes 1478 so that the gases being vented can leave in one lumen (for example, the smaller lumen 1486) and the gases entering from the gases port 106 can enter the cannula lumen via the other lumen (for example, the bigger lumen 1484).

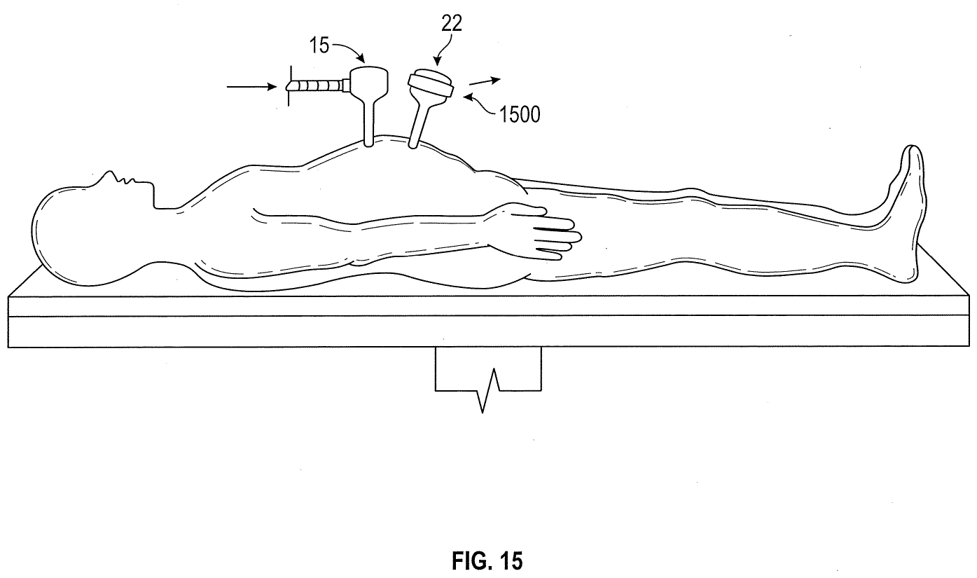

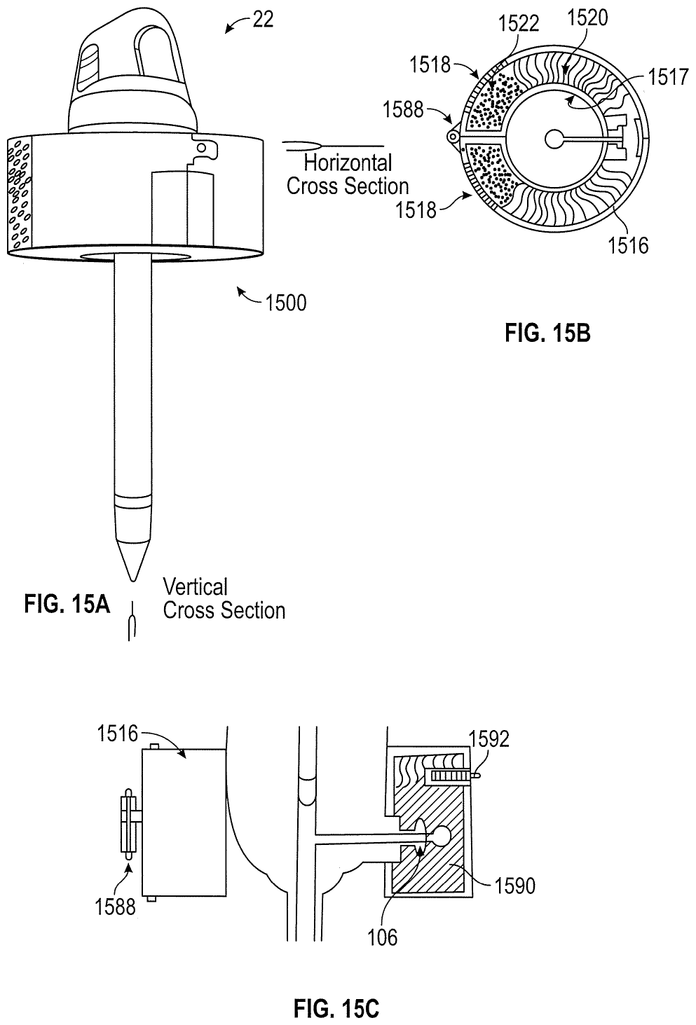

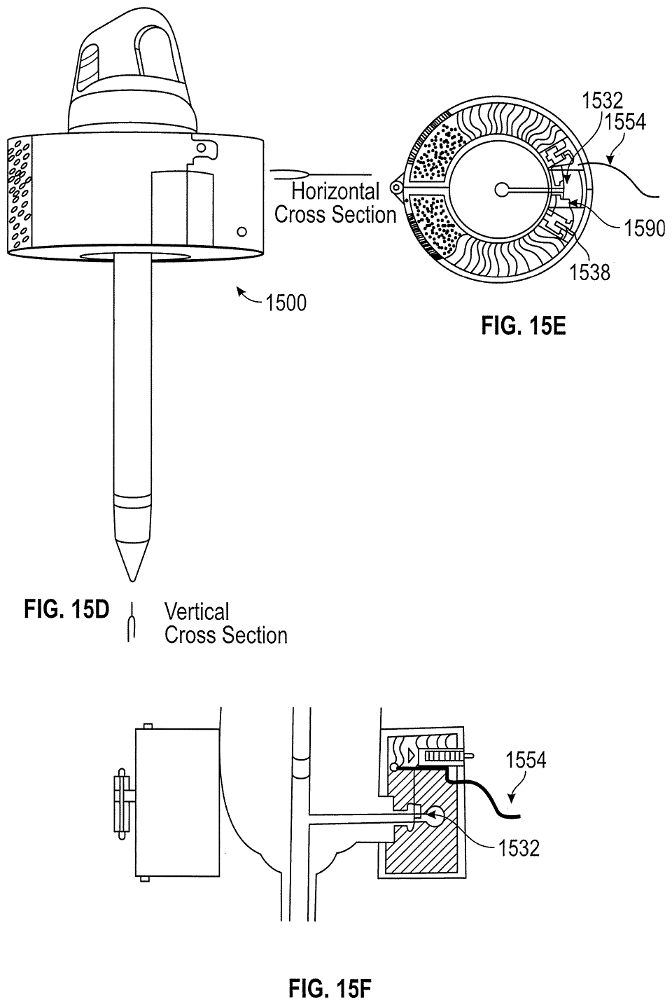

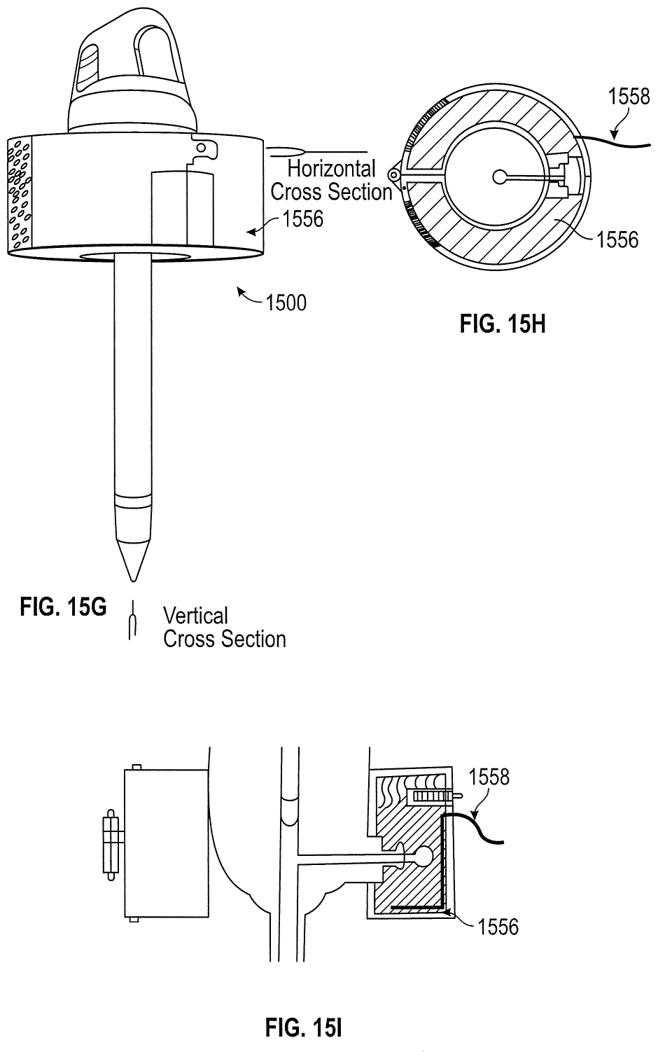

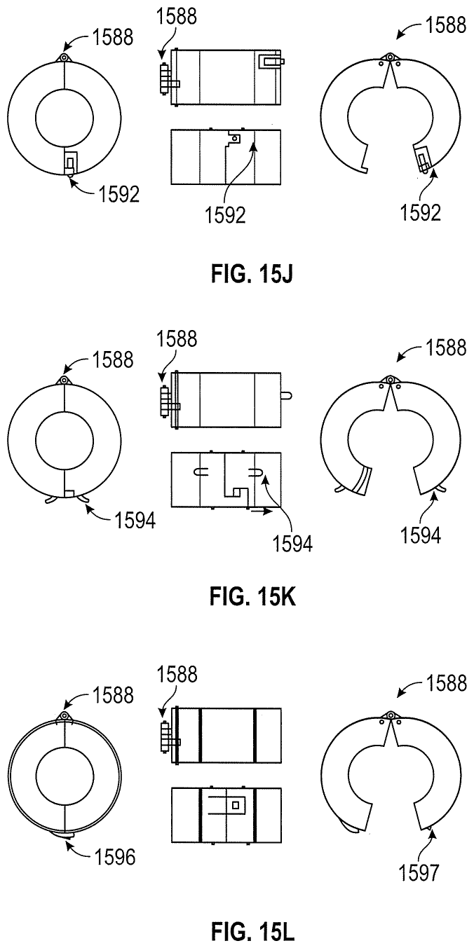

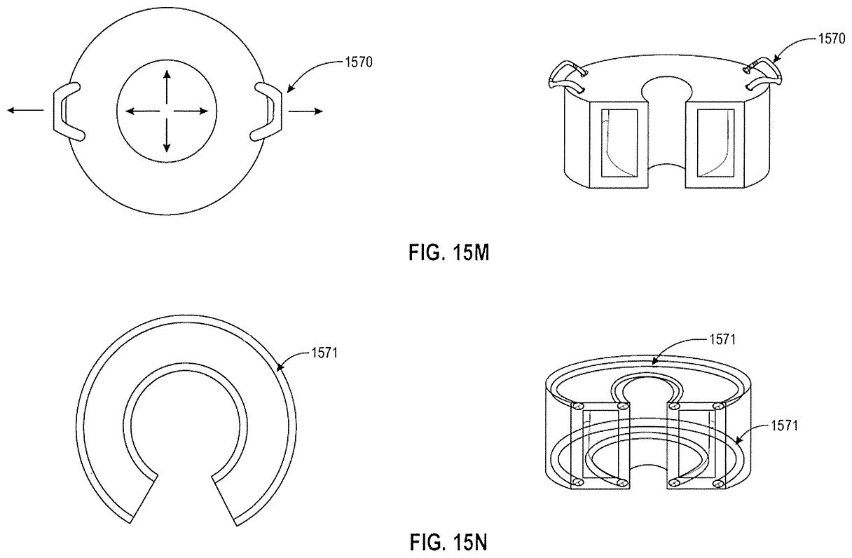

FIGS. 15 and 15A-15N illustrate examples of a hinged leak device 1500 configured to be coupled to a venting cannula 22. As shown in FIG. 15, the venting cannula 22 can be a different cannula than the insufflation cannula 15 configured to deliver insufflation fluid into and/or allow a medical instrument to be inserted into the surgical cavity within the patient's body.

The hinged device 1500 can clamp around a portion of the upper housing 102 of the cannula 22. The hinged leak device 1500 can include a lumen configured to receive a portion of the upper housing 102 and/or a portion of the elongate shaft 104 so that the leak device 1400 circumferentially surrounds a portion of the upper housing 102 and/or a portion of the elongate shaft 104. The hinged device 1500 can include two halves. A hinge mechanism 1588 can connect the two halves and allow the device 1500 to be opened and closed using a latch (for example, operated by spring) 1592 or other locking mechanisms to lock the two halves in place. The latch can be made of aluminum or other metals, or plastics.

The hinged leak device 1500 can include gases impermeable walls 1516 (for example, being made of plastic, for example, acrylonitrile butadiene styrene (ABS) and the like) with a plurality of vents 1518 configured to vent the gases. In some configurations, the curved inner 1517 and outer 1516 walls of the device 1500 can have a thickness of, for example, about 0.1 mm to about 3 mm, or about 1 mm to about 2 mm, or thinner. In some configurations, the curved inner 1517 and outer 1516 walls of the device 1500 can have a thickness of about 1 mm. The divider wall between the two halves can have a thickness of about 2 mm. As shown in FIG. 15B, the vents 1518 can be located on two sides of the hinge mechanism 1588. When the device 1500 clamps around the cannula 22, the gases port 106 of the cannula 22 can be located generally opposite the hinge mechanism 1588. The gases port 106 can be surrounded by a rubber or elastomeric (for example, silicone) material 1590 in the device 1500 to seal around the gases port 106. The device 1500 can receive gases from the gases port 106 of the cannula 22. The gases can be guided by a gases path in the rubber material 1590 to diffuse through particulate filters 1520 (for example, ULPA filters). The filters 1520 can be located on both sides of the rubber material 1590 and can extend about ⅔ of a circumference (or other lengths) of the device 1500. Odor filters (for example, carbon filters) 1522 can be located between the particulate filters 1520 and the vents 1518. The vents 1518 can allow the filtered gases and/or smoke to be released to the atmosphere.

The device 1500 can optionally include one or more mechanical or passive venting valves in a gases pathway in the device 1500, for example, to replace and/or be used in combination with small orifices at the vents 1518. As shown in FIGS. 15D-15F, the valves can also optionally include electrical valves 1538. The electrical valves 1538 can be in electrical communication with a control system (for example, a PCB) via a wire 1554 so as to actuate the electrical valves 1538. Examples of valves 1538 used can include an umbrella pressure relief valve, a solenoid gate valve, a standard pressure relief valve, and/or a diaphragm pressure relief valve as described above. In some configurations, the valves 1538 can include safety valves that are configured to vent at about 10 mmHg to about 50 mmHg, or about 50 mmHg. As shown in FIG. 15E, each valve 1538 can include a small restricting gases path in communication with the path in the rubber material 1590 to direct the gases from the cannula 22 through the paths in the valves 1538. The valve(s) 1538 can be selectively opened or closed (for example, to control the venting or leak rate).

As shown in FIG. 15F, the wire 1554 can extend between the rubber material 1590, the wall 1516 of the device, and the filter 1520. The wire 1554 can be connected to a control system of the insufflator or the humidifier unit or an external controller. The wire 1554 can also split to connect to a flow rate and/or smoke sensor 1532, or any other suitable sensors, for example, a humidity sensor, which can be moulded into the wall of the rubber or elastomeric material 1590 next to the gases port 106. The wire 1554 may include a single wire or a multi wire cable. In some configurations, the multi wire cable may include an insulating cover. The cable may include multiple communication channels to allow heating and/or sensing signals to be transmitted to a controller, for example, the humidifier controller. The humidifier may supply power to the heater wire.

As shown in FIGS. 15G-15I, the hinged leak device 1500 can include a heating element (for example, a flexible PCB heater) 1556. The heating element 1556 can be located on or in a groove in a bottom of the device 1500 or be moulded into the device 1500. As the device 1500 can be made of two halves, a connecting pin or other feature can be used to bridge the gap between two separate heaters 1556 so that the heaters can act as a single heating element. The electrical connection between the two heating elements 1556 can be achieved by the connecting pin or other feature. The heating element 1556 can be configured to heat the filters 1520, 1522 and/or the gases passing through the device 1500 before the gases reach the filter(s) 1520, 1522. The heated gases can reduce and/or prevent condensation and clogging in the filter(s), which can extend the life of the filter and aid in maintaining efficiency of the filter. The heating element 1556 can be powered by the PCB via a wire connection 1558.

FIGS. 15J-15N illustrate methods of securing the hinged leak device 1500 to the cannula. FIG. 15J illustrate the hinge and latch mechanism described above. When closing the latch, a spring-biased pin 1592 can be pushed in and then released to lock the device 1500 in place. As shown in FIG. 15K, the device 1500 can have a squeeze lock and hinge mechanism. The L-shaped locks 1594 can be squeezed to release tension and unlock the two halves of the device 1500. FIG. 15L illustrate a clip lock and hinge mechanism. A user can push a fit clip 1596 onto an inner clip 1597 to lock the two halves of the device 1500 and push against the inner clip 1597 to release the two halves. FIG. 15M illustrates the hinged leak device 1500 made from a flexible material (for example, rubber, nitrile, or other flexible materials, for example, flexible materials that can expand to, for example, at least 200% of their original sizes). The device 1500 may not form a complete circle to promote ease of access for a user to deform the device 1500 to a desired shape. The inner diameter of the device 1500 can have an initial internal diameter smaller than the outer diameter of the cannula (for example, the cannula upper housing). In some configurations, the inner diameter of the device 1500 can have an initial diameter that is, for example, about 5% to about 50%, or about 20%, or about 25%, or about 30%, less than the outer diameter of the cannula. The device 1500 can have handles 1570 located on generally opposing ends of the device 1500. A user can pull down the handles 1570 to expand the internal diameter of the device 1500 to attach the device 1500 to the cannula. The restoring forces or internal tension of the flexible material tries to return the internal diameter of the device 1500 to its original size, thereby maintaining the device 1500 on the cannula.

FIG. 15N illustrates a combination of a plurality of (for example, four or any other number) wires 1571 in a hinged leak device 1500 made of the flexible material described above. The wires 1571 can be made of a malleable metal. The device 1500 may not form a complete circle to promote ease of access for a user to deform or mould the device 1500 to a desired shape. The wires 1571 can be placed along or near the inner and outer edges of the device 1500 to provide structural support to the device 1500 in any moulded position.

Additional Examples of a Venting Attachment

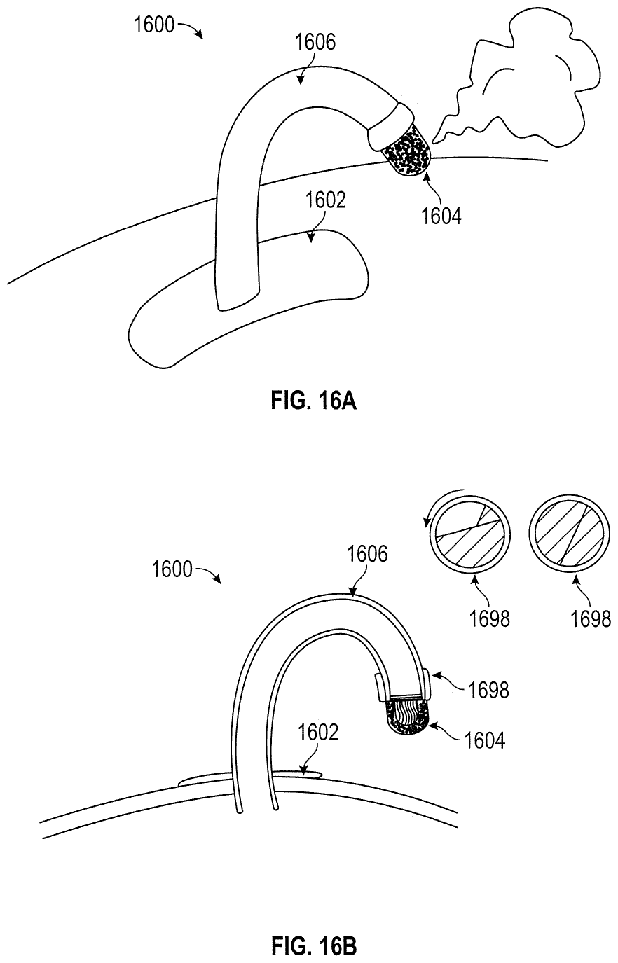

FIGS. 16A and 16B illustrates a standalone leak device 1600 configured to be used without being coupled to a cannula. The leak device 1600 can have a skin contacting end 1602 and a venting end 1604. The skin contacting end 1602 can include a flexible seal configured to adhere to the skin (for example, like a plaster) over a wound opening providing access to a surgical cavity. One end of a tube 1606 (for example, a thin flexible tube) can be pushed through the flexible material and into the surgical cavity. An opposite end of the tube 1606 can be coupled to the venting end 1604. The venting end 1604 can contain the particulate and/or odor filters 1620, 1622, for example, the ULPA filter and the carbon filter. The venting end 1604 can include a plurality of vents. A twistable toggle 1698 can control a size of the vent so as to control the vent rate.

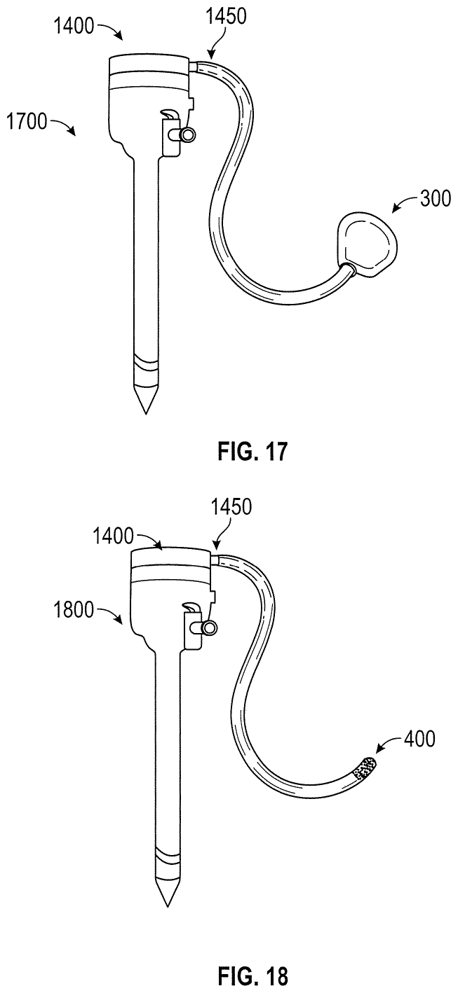

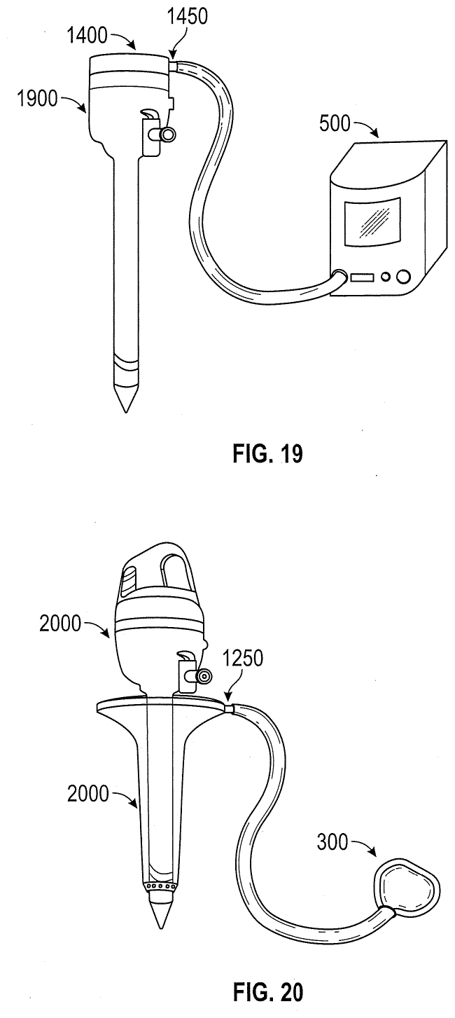

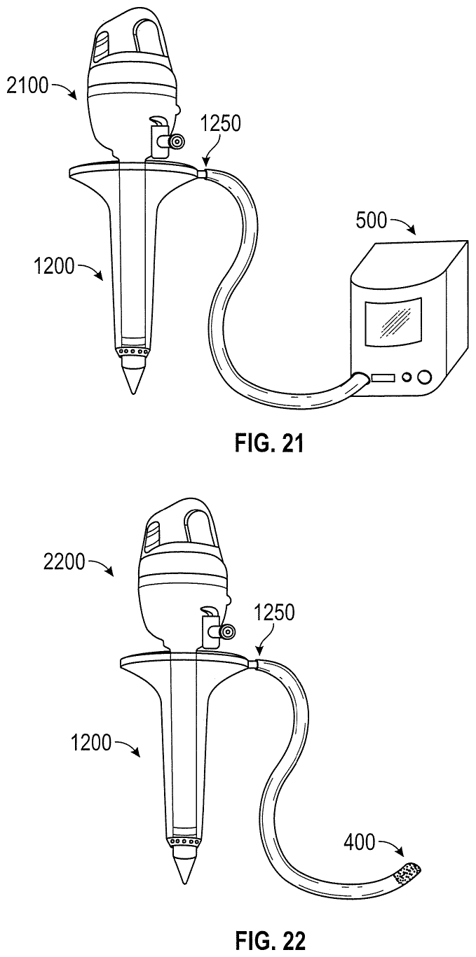

FIGS. 17-22 illustrate leak devices incorporating more than one of the leak device examples disclosed herein. The leak device 1700 in FIG. 17 includes an insertable filter device portion 1400 and a suspended filter device portion 300, with the suspended filter device portion 300 coupled to the proximal vents 1450 of the insertable filter portion 1400. The leak device 1800 in FIG. 18 includes an insertable filter device portion 1400 and a suspended filter device portion 400, with the suspended filter device portion 400 coupled to the proximal vents 1450 of the insertable filter device portion 1400. The leak device 1900 in FIG. 19 includes an insertable filter device portion 1400 and a filter device portion 500 with a housing having any suitable shape, such as the rectangular-shaped filter device portion 500 coupled to the proximal vents 1450 of the insertable filter device portion 1400 as shown, or having a shape that is round, curved, elliptical, polygonal, or the like. The leak device 2000 in FIG. 20 includes a shaft sleeve leak device portion 1200 and a suspended filter device portion 300, with the suspended filter device portion 300 coupled to the proximal vents 1250 of the shaft sleeve leak device portion 1200. The leak device 2100 in FIG. 21 includes a shaft sleeve leak device portion 1200 and a rectangular-shaped filter device portion 500, with the rectangular-shaped filter device portion 500 coupled to the proximal vents 1250 of the shaft sleeve leak device portion 1200. The leak device 2200 in FIG. 22 includes a shaft sleeve leak device portion 1200 and a suspended filter device portion 400, with the suspended filter device portion 400 coupled to the proximal vents 1250 of the shaft sleeve leak device portion 1200.

The leak devices in FIGS. 17-22 can incorporate features of both types of leak device portions, for example, the cannula connecting features and the filters. In some configurations, the insertable filter and/or shaft sleeve portions of the leak devices may not include filters so that the filters in the other leak device portion, for example, the suspended filter or rectangular-shaped filter device portions, can be used to filter the vented gases and/or smoke. The cannula coupled to those leak device examples can have the gases port and gases delivery lumen unobstructed for delivering the insufflation fluid.

As shown in FIG. 23, a cannula 15 (for example, a double-lumen cannula) can also be coupled to a recirculation line 2300. The cannula 15 can receive insufflation fluid from the humidifier unit 3 via the gases delivery tube 13. The cannula 15 can vent the gases and/or smoke (for example, filtered gases and/or smoke) through the recirculation line 2300 back to the humidifier unit 3 or surgical system. The gases can also be filtered by a filter system in the humidifier unit 3 before re-delivery into the surgical cavity.

In some configurations, a filter for example, those described and illustrated herein can comprise multiple filter elements. The multiple filter elements can be arranged in fluid communication with the venting passage/venting lumen. The filter elements can be placed within a venting gases path. The filter elements can be arranged in series such that the vented gases/smoke travels through two or more filter elements. Different types of filters can be used in the multi-filter elements. For example, a carbon filter and a UPLA filter can be used to filter out particulate matter and any potentially harmful substances in the vented gases.

In some configurations, a venting gases cannula for example, those described and illustrated herein may include one or more heating elements. The heating elements may be located within one, two, or more lumens. The heating elements may extend a partial length or the entire length of the lumens. The heating elements are configured to heat the gases being delivered to the surgical cavity to maintain the temperature of the cavity at a desired value. Further heating the gases prevents condensation of the insufflation fluid and vented gases. Further heating the gases can also reduce and/or prevent fogging and/or condensation on any instruments e.g., scopes. Furthermore, one or more heating elements can be in communication or in contact with one or more filter elements to heat the filter elements. Heating the filter elements prevents clogging and condensation in the filter. Additionally, the heating elements may also be structured and configured to heat one or more valves in order to heat the vents or valves. Heating these portions reduces and/or prevents condensation forming in the vents and clogging the vents.

### Terminology

Examples of medical gases delivery systems and associated components and methods have been described with reference to the figures. The figures show various systems and modules and connections between them. The various modules and systems can be combined in various configurations and connections between the various modules and systems can represent physical or logical links. The representations in the figures have been presented to clearly illustrate the principles and details regarding divisions of modules or systems have been provided for ease of description rather than attempting to delineate separate physical embodiments. The examples and figures are intended to illustrate and not to limit the scope of the inventions described herein. For example, the principles herein may be applied to a surgical humidifier as well as other types of humidification systems, including respiratory humidifiers. However, the humidification systems and methods may also optionally not involve a patient's respiratory system and may not be placed within a portion of the respiratory tract (for example, nose, mouth, trachea, and/or bronchi).

As used herein, the term “processor” refers broadly to any suitable device, logical block, module, circuit, or combination of elements for executing instructions. For example, the controller 8 can include any conventional general purpose single- or multi-chip microprocessor such as a Pentium® processor, a MIPS® processor, a Power PC® processor, AMD® processor, ARM® processor, or an ALPHA® processor. In addition, the controller 122 can include any conventional special purpose microprocessor such as a digital signal processor or a microcontroller. The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein, or can be a pure software in the main processor. For example, logic module can be a software-implemented function block which does not utilize any additional and/or specialized hardware elements. Controller can be implemented as a combination of computing devices, e.g., a combination of a DSP and a microprocessor, a combination of a microcontroller and a microprocessor, a plurality of microprocessors, one or more microprocessors in conjunction with a DSP core, or any other such configuration.

Data storage can refer to electronic circuitry that allows data to be stored and retrieved by a processor. Data storage can refer to external devices or systems, for example, disk drives or solid state drives. Data storage can also refer to fast semiconductor storage (chips), for example, Random Access Memory (RAM) or various forms of Read Only Memory (ROM), which are directly connected to the communication bus or the controller. Other types of data storage include bubble memory and core memory. Data storage can be physical hardware configured to store data in a non-transitory medium.

Although certain embodiments and examples are disclosed herein, inventive subject matter extends beyond the specifically disclosed embodiments to other alternative embodiments and/or uses, and to modifications and equivalents thereof. Thus, the scope of the claims or embodiments appended hereto is not limited by any of the particular embodiments described herein. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is otherwise understood with the context as used in general to convey that an item, term, etc. may be either X, Y or Z. Thus, such conjunctive language is not generally intended to imply that certain embodiments require at least one of X, at least one of Y and at least one of Z each to be present. As used herein, the words “about” or “approximately” can mean a value is within ±10%, within ±5%, or within ±1% of the stated value.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may comprise connected logic units, such as gates and flip-flops, and/or may comprised programmable units, such as programmable gate arrays, application specific integrated circuits, and/or processors. The modules described herein can be implemented as software modules, but also may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

1. A venting attachment to a surgical cannula for venting one or both of gases or smoke from a surgical cavity, wherein the surgical cannula comprises a gases port, the venting attachment comprising:
a cannula connecting component configured to connect to a portion of the surgical cannula inserted into the surgical cavity;
a venting component configured to vent one or both of gases or smoke from the surgical cavity at a predetermined rate, an orifice of the cannula connecting component having a size that allows the venting at the predetermined rate;
a venting gases pathway extending from the cannula or the surgical cavity to the venting component; and
a filter component configured to filter the one or both of gases or smoke before leaving the venting component, wherein the filter component is located in the venting gases pathway and adjacent the venting component,
wherein the cannula connecting component comprises a connector that forms a seal around an outer surface of a gases port of the surgical cannula when the cannula connecting component and the gases port are coupled.

2.-42. (canceled)

43. The venting attachment of claim 1, wherein the connector is a Luer lock connector.

44. The venting attachment of claim 1, wherein the seal between the cannula connecting component and the gases port is the only seal between the cannula connecting component and the gases port.

45. The venting attachment of claim 1, wherein the cannula connecting component is configured to receive and guide the gases port during insertion into the cannula connecting component.

46. The venting attachment of claim 1, wherein the cannula connecting component comprises an opening, a neck region, and/or a confined area.

47. The venting attachment of claim 46, wherein the neck region is immediately adjacent the opening and the confined area.

48. The venting attachment of claim 46, wherein the neck region is configured to deform to allow passage of the gases port.

49. The venting attachment of claim 48, wherein the gases port comprises a flanged end portion and the neck region is configured to deform to allow passage of the flanged end portion.

50. The venting attachment of claim 46, wherein the confined area is configured to receive and retain the gases port when the cannula connecting component and the gases port are coupled.

51. The venting attachment of claim 50, wherein the gases port comprises a flanged end portion and the confined area is configured to receive and retain the flanged end portion when the cannula connecting component and the gases port are coupled.

52. The venting attachment of claim 46, wherein the neck region conforms around an outer surface of the gases port to form the seal when the cannula connecting component and the gases port are coupled.

53. The venting attachment of claim 52, wherein the seal is formed only between the neck region and the outer surface of the gases port.

54. The venting attachment of claim 52, wherein the gases port comprises a shaft portion and the neck region conforms around an outer surface of the shaft portion to form the seal when the cannula connecting component and the gases port are coupled.

55. The venting attachment of claim 54, wherein the seal is provided along a length of the shaft portion.

56. The venting attachment of claim 1, wherein the venting component is configured to vent the gases and/or smoke at a rate between 1 L/min and 10 L/min.

57. The venting attachment of claim 56, wherein the venting component is configured to vent the gases and/or smoke at a rate between 5 L/min and 7 L/min.

58. The venting attachment of claim 56, wherein the venting component is configured to vent the gases and/or smoke at a rate of 4 L/min.

59. A venting attachment to a surgical cannula for venting one or both of gases and/or smoke from a surgical cavity, the venting attachment comprising:
a venting component configured to vent one or both of gases or smoke from the surgical cavity at a predetermined rate;
a venting gases pathway extending from the cannula or the surgical cavity to the venting component; and
a filter component configured to filter the one or both of gases or smoke before leaving the venting component, wherein the filter component is located in the venting gases pathway and adjacent the venting component,
wherein the venting component is at least partially inserted through a surgical opening leading to the surgical cavity.

60.-82. (canceled)

83. A venting attachment to a surgical cannula for venting one or both of gases or smoke from a surgical cavity, the surgical cannula comprising a gases port and an inlet, the venting attachment comprising:
a venting component configured to be inserted through the inlet of the surgical cannula, the venting component configured to vent one or both of gases or smoke from the surgical cavity at a predetermined rate;
a venting gases pathway extending from the cannula or the surgical cavity to the venting component; and
a filter component configured to filter the one or both of gases or smoke before leaving the venting component, wherein the filter component is located in the venting gases pathway.

84.-104. (canceled)

105. A venting attachment to a surgical cannula for venting one or both of gases or smoke from a surgical cavity, wherein the surgical cannula comprises a gases port, the surgical cannula further having a proximal end and a distal end, the proximal end including an inlet and the distal end of the surgical cannula configured to be inserted into the surgical cavity, the venting attachment comprising:
a cannula connecting component configured to connect to the gases port or an inlet of the surgical cannula;
a venting component configured to vent one or both of gases or smoke from the surgical cavity at a predetermined rate;
a venting gases pathway extending from the cannula or the surgical cavity to the venting component; and
a filter component configured to filter the one or both of gases or smoke before leaving the venting component, wherein the filter component is located in the venting gases pathway and adjacent the venting component,
wherein the venting component at least partially surrounds the proximal end of the surgical cannula.

106.-127. (canceled)

128. A venting attachment to a surgical cannula for venting one or both of gases or smoke from a surgical cavity, the venting attachment comprising:
a leak device including a flow restriction within a passage of the leak device, wherein the flow restriction is shaped and dimensioned to control a venting flow rate such that the venting flow rate is equal to or less than the flow rate of gases delivered into the surgical cavity,
the leak device further comprising a cannula connector.

129. A surgical cannula for providing insufflation gases to a surgical cavity and venting from the surgical cavity, comprising:
an upper housing including an opening;
an elongate shaft extending from the upper housing;
a first lumen in the elongate shaft configured to receive the insufflation gases from a gases source;
a second lumen in the elongate shaft configured to vent gases from the surgical cavity, the first and second lumens in fluidic communication with the opening; and
a leak device comprising:
a cannula connecting component configured to connect to a portion of the cannula;
a venting component configured to allow one or both of gases or smoke to exit the surgical cavity;
a venting gases pathway extending from the cannula to the venting component, the venting gases pathway being in fluidic communication with the second lumen; and
a filter component configured to filter the one or both of gases or smoke before leaving the venting end, wherein the filter component is located in the venting gases pathway and adjacent the venting component.

130.-144. (canceled)

145. A method of venting gases from a body cavity, comprising:
inserting a cannula into the body cavity, the cannula comprising a venting attachment, wherein the body cavity is configured to receive an insufflation gas from said cannula or another cannula;
filtering one or both of the insufflation gases or surgical smoke through a filter within the venting attachment; and
venting the one or both of filtered insufflation gases or surgical smoke from the body cavity through the venting attachment.

146.-149. (canceled)
